(19) United States
(12) Patent Application Publication
(10) Pub. No.: US 2010/0105857 A1
(43) Pub. Date: Apr. 29, 2010

(54) DENDRIMERIC PLATFORM FOR CONTROLLED RELEASE OF DRUGS

(76) Inventors: Gary Gellerman, Neve Yam (IL); Michael Firer, Karnei Shomron (IL)

Correspondence Address: The Law Office of Michael E. Kondoudis, 888 16th Street, N.W., Suite 800, Washington, DC, 20006, US

(21) Appl. No.: 12/444,118

(22) PCT Filed: Oct. 11, 2007
(86) PCT No.: PCT/IL2007/001225

§ 371 Date: Nov. 23, 2009

(30) Foreign Application Priority Data
Oct. 16, 2006 (IL) 178645

(51) Int. Cl. C08G 69/00

(52) U.S. Cl. 528/271

(57) ABSTRACT

A multifunctional molecular platform is provided, for covalent binding of two or more therapeutic or diagnostic agents, and for their sequential release in a biological environment near desired target sites. The platform is used in the preparation of pharmaceutical compositions for treating abnormal cell proliferation, infections, and inflammation.

### FIELD OF THE INVENTION

The present invention relates to the field of drug delivery by means of dendrimers, and particularly to new compositions comprising dendrimeric structures and use thereof for controlled release of a plurality of drugs.

### BACKGROUND OF THE INVENTION

Prerequisites of an efficient disease treatment include employing an active agent at right place at right time, which makes the drug delivery as important as the drug activity. Among the means considered for drug delivery there are also dendrimers—highly branched oligomeric or polymeric structures. A dendrimer is created from a low molecular core having at least two attachment points, and a monomer unit having at least three attachment points, by covalently linking said monomer units to all the attachment points on the core, thereby obtaining a dendrimer of the first generation; each of the linked monomer units provides at least two free attachment points for eventual further growth, and for providing a dendrimer of the second generation. The number of built-in monomer units in the growing dendrimer at least doubles in each generation, leading gradually to a tree-like regular structure (dendros being tree in Greek). The attachment points, embodying in fact the branching points of the dendrimer topology, may be realized by a variety of reactive chemical groups; the free attachment points of the highest generation, “leaves of the dendrimer tree”, represent a pool of terminal groups for eventual further chemical interactions. An agent to be delivered may be physically encapsulated within a dendrimer, or may be bound to it by noncovalent interactions, or may be covalently linked to said terminal groups [see, e.g., Zeng F. & Zimmerman S. C.: Chem. Rev. 97 (1997) 1684-712; Svenson S. & Tomalia D. A.: Advanced Drug Deliv. Rev. 57 (2005) 2106-29)].

Different diseases differ in the location and type of the tissues to be targeted, in the chemical nature of the drugs to be delivered, and in the required delivery regimen; the corresponding pharmacokinetic issues involve possible interactions among the components, dosing and stability of the active agents, as well as their temporally and spatially optimal release, necessitating to develop an assortment of various carriers. For example, U.S. Pat. No. 5,714,166 relates to a dendrimer coupled to at least one bioactive agent, particularly the agent being a biological response modifier. U.S. Pat. No. 5,830,986 provides a method for synthesizing a dendrimer based on polyethylene oxide for binding a biologically active molecule. U.S. Pat. No. 6,020,457 relates to dendritic polymers for drug delivery, containing a disulfide moiety in the core. US 2002/0071843 relates to a targeting therapeutic agent comprising a targeting entity which binds to a site of pathology, a linking factor, such as a dendrimer, and a therapeutic entity, the factor eventually binding additional materials. US 2003/0180250 claims a dendrimer complexed with an anti-inflammatory drug. WO 2004/019993 discloses a self-immolative dendrimer that releases many active moieties upon a single activating event. US 2004/0228831 describes a polymeric drug conjugate comprising one or more biologically active agents conjugated via an enzymatically cleavable linker, for targeting a diseased tissue.

The previously described dendrimers do not relate to independent release of two or more therapeutic or diagnostic agents; therefore, and also in view of the continuing need of new diversified dendrimers for drug delivery, it is an object of this invention to provide novel dendrimers for drug delivery.

It is another object of this invention to provide dendrimers for drug delivery, enabling programmed release of at least two therapeutic or diagnostic agents.

It is still another object of this invention to provide dendrimers for drug delivery for use in programmed, sequential, multi-drug release at a target site.

It is further an object of this invention to provide a dendrimer-based platform with at least two types of active attachment points for coupling at least two different agent or label molecules for use in programmed, sequential, multi-drug release at a target site.

Other objects and advantages of present invention will appear as description proceeds.

### SUMMARY OF THE INVENTION

The present invention provides a multifunctional platform for covalent binding of at least two different therapeutic or diagnostic agents and for their sequential release at a target site in a biological environment, said platform being a molecular structure that has i) at least two reactive terminal groups (called attachment moieties), of at least two different kinds, through which said at least two different agents are bound, forming at least two types of linkage moieties, resulting in at least two different types of cleaving kinetics under the conditions of said biological environment; and ii) an additional terminal group (called carrier moiety) differing from said attachment moieties, through which a recognition structure, called carrier, is bound, wherein said carrier assists in delivering at least one of said therapeutic or diagnostic agents to said target site. In a preferred embodiment, the platform of the invention is a molecular structure that has at least four attachment moieties, of at least two different kinds, through which said at least two different agents are bound, forming at least two types of linkage moieties, resulting in at least two different types of cleaving kinetics under the conditions of said biological environment, wherein each of said agents is bound to the platform as at least one pair of molecules. The platform of the invention preferably comprises, beside a carrier moiety, numerous copies of molecular substructures, wherein each substructure is capable of binding and releasing differentially at least two therapeutically useful agents. Said carrier assists in delivering at least one of said therapeutic or diagnostic agents to a desired site of action. A multifunctional platform according to the invention has preferably more than two attachment moieties of each kind, and may bear more than two kinds of attachment moieties. Said moieties on the platform according to the invention may comprise reactive groups, such as amino, or blocked reactive groups, such as amino-Teoc. The platform of the invention may be illustrated by a structure selected, for example, from formulae 7-5, 7-7, 7-10, 7-13, 8-1, 8-2, 9-1, 9-2, 9-4, 10-1, 10-6, 11, 11-3, 11-8, and 11-9. The platform may have a general structure depicted by formulae 13-1, 13-2, 13-3, 13-4, 13-5, 13-6, 13-7, 13-8, 13-9, 13-10, 13-11, 13-12, and 13-13. In one embodiment, a multifunctional platform according to the invention has structure 14 as follows:

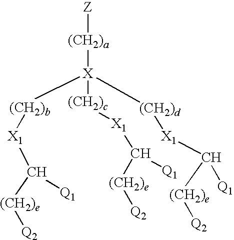

wherein X represents carbon atom, or substituted heterocyclic or aromatic ring selected from benzene, naphthalene, diphenyl, phenylbenzyl;
Z is a reactive group selected from —COOH, —NH2, —NHalkyl, —OH, —SSH, SH, and —NHNH2;
a, b, c, d, and e are integers independently selected from 1 to 5;
X₁ is selected from —NH—, —NHCO—, and —CONH—, —O—, and —S—; and
Q₁ and Q₂ are groups independently selected from NHR, NHNR, COOR, OR, SR, S—SR, POₙR wherein n is 1-3, wherein R is selected from H, alkyl, aryl, and blocking groups, wherein said blocking group may be for example selected from Alloc, Fmoc, Boc, Teoc, TFA, and Dde, for NHR or NHNHR; from Acm, Trityl and s-tBu for SR or SSR, and from Me, Allyl, Benzyl and Fluorenemethylene for COOR, which blocking groups can be replaced by two different drug molecules, and wherein said reactive group Z couples said multifunctional platform to a carrier.

A multifunctional platform according to the invention may comprise at least two covalently coupled drugs, as illustrated, for example, by structures 2-1, 3-1, 4-1, 5-8, 6-1, 7-10, 8-7, 9-6, 10-4, and 11-11. Said sequential release of said agents may be initiated or stimulated by different conditions at different sites of said biological environment, possibly comprising one or more hydrolytic enzymes, or changes in pH, wherein the differences in different tissues or subcellular compartments may be involved. A multifunctional platform according to the invention may comprise coupled drugs, wherein the drugs are linked via moieties comprising at least one item selected from ester, amide, secondary amide, carbamate, thiocarbamate, urea, thiourea, ether, thioether, and —S—S— group.

The invention relates to a pharmaceutical composition comprising a platform according to the invention, as described above. The invention further relates to a pharmaceutical composition comprising a drug bonded to a platform according to the invention. Said drug may involve any compound useful in therapy or diagnosis, that is capable of being coupled to the platform directly or after derivatizating the compound. The compound may be activated before coupling, using known methods. Said composition may be used in treating diseases in which the application of more than one drug is indicated, for example diseases selected from diseases associated with abnormal cell proliferation, diseases associated with microbial or viral infections, diseases associated with inflammation and autoimmune diseases.

The platform according to the invention may be a simple dendrimer-like structure, or it may be highly branched dendrimeric structure comprising a plurality of attachment points which can be used for binding drugs or for further branching of the structure. Said highly branched dendrimeric structure may be obtained from a platform of the invention by employing said attachment moieties, instead of for binding drug molecules, for binding a linker containing at least two additional attachment moiety, the additional moiety being the same type or different than the original moiety.

The invention provides a method for preparing the multifunctional platform of claim 1, comprising the steps of i) providing a molecular structure comprising reactive groups of at least two different kinds, the location of the to groups defining attachment points on said structure, the group kinds independently selected from —YₘPₘ, wherein Yₘ is a radical comprising one of —NH, —O, —S, —SS, —COO, —NHNH, —N-alkyl-NH, -Ph-NH, -Ph-CH2-NH, -Ph-O, -Ph-S, —N-alkylene, —N-cycloalkylene, or POₙ wherein n is from 1 to 3, and wherein Pₘ is a blocking group used in SPOC; ii) contacting said structure of step i) in a solution with a resin capable of reacting with one kind of said reactive groups, thereby linking the structure through one of the attachment points to the resin and immobilizing it; contacting said immobilized structure of step ii) with at least two different drugs under conditions enabling the replacement of two remaining kinds of said blocking groups by the molecules of said drugs, thereby obtaining the immobilized platform loaded with at least two drugs; and iv) releasing said loaded platform from the resin and binding it through said attachment point of step iii) to a carrier. Said Yₘ may be a radical selected from the group consisting of —NH, —(CH2)ₙNH, —O, —(CH2)ₙO, —S, —(CH2)ₙS, —SS, —(CH2)ₙSS, —COO, —(CH2)ₙCOO, —NHNH, (CH2)ₙNHNH, —N-alkyl-NH, —(CH2)ₙN-alkyl-NH, -Ph-NH, —(CH2)ₙPh-NH, -Ph-CH2-NH, (CH2)ₙPh-CH2-NH, —N-alkyl, —(CH2)ₙN-alkylene, or —N-cycloalkylene, —(CH2)ₙN-cycloalkylene, -Ph-O, -Ph-S, —PO, —PO₂, and —PO₃. Said Pₘ may be a blocking group selected from Fmoc, Alloc, Teoc, Boc, Dde, Phthalimide, Treoc, and TFA when Yₘ is a radical comprising —NH; Allyl, Benzyl, Dimethoxybenzyl, Acetyl, Fluorenemethylene, t-Bu, Trityl, when Yₘ is a radical comprising —O;

S-tBu, tBu, Trityl, Acm, when Yₘ is —S; and Me, Allyl, Benzyl, Dimethoxybenzyl, Fluorenemethylene, t-Bu, when Yₘ is a radical comprising —COO. Said carrier is a molecular structure covalently linked to said platform, assisting in delivering a therapeutic or diagnostic agent to the desired site of action in a tissue, either targeting said tissue or stabilizing said agents during their transport to the tissue. Said carrier molecular structure may be a molecule or a part thereof selected from protein, peptide, phospholipid, polysaccharide, nucleic acid or a structural mimic thereof, such as a peptide nucleic acid (PNA) and biodegradable polymer. Said carrier molecular structure may be a molecule or a part thereof having high affinity to a tissue to be treated. Said carrier molecular structure may recognize or be recognized by a treated tissue, or cells involved in the disease, or it may interact with a regulation cascade in vivo, thereby initiating processes supporting intended therapeutic goals. Said carrier may be a biodegradable polymer.

### BRIEF DESCRIPTION OF THE DRAWINGS

The above and other characteristics and advantages of the invention will be more readily apparent through the following examples, and with reference to the appended drawings, wherein:

FIG. 1. is a reaction scheme showing the preparation of a simple model platform for binding two agents, one molecule each;

FIG. 2. is a reaction scheme showing the preparation of a platform for binding two agents, two molecules each;

FIG. 3. is a reaction scheme showing the preparation of a platform for binding two agents, two molecules each;

FIG. 4. is a reaction scheme showing the preparation of a platform for binding two agents, two molecules each;

FIG. 5. is a reaction scheme showing the preparation of a platform loaded with two agents; 5A showing the preparation of active platform with the attachment moieties; and 5B showing the coupling of the drugs;

FIG. 6. is a reaction scheme showing the preparation of a platform for binding two agents, two molecules each (“CA” stands for “commercially available);

FIG. 7. demonstrates some principles involved in creating platforms of the invention; 7A being a schematic representation of two platform topologies; 7B illustrating a dendrimer obtained from simpler structures; 7C showing a generalized structure based on 3,4,5-trihydroxybenzoicacid (THB); 7D demonstrating loading drugs onto a THB platform; 7E and 7F present two examples of simple platforms and list reactive groups useful in creating platform intermediates, to be activated with blocking groups, and later loaded with drugs (the cross point representing a carbon atom, and the “D and L” reminding a possible isomerism at the point);

FIG. 8. demonstrates creating a dendrimeric platform based on benzene, built from pyromellitic dianhydride; 8A being a reaction scheme; 8B showing different attachment moieties for two drugs;

FIG. 9. is a reaction scheme showing the preparation of a platform for binding three agents, twelve molecules each; 9A showing creating the platform with attachment moieties; 9B showing coupling the activated drugs; and 9C showing the preparation of various moieties for attaching the platform to a solid support before drug loading (for various attachment modes to the carrier);

FIG. 10. is a reaction scheme showing the preparation of a platform comprising photocleavable linkers; 10A showing the creation of the intermediate platform with the linkers; 10B showing the binding the intermediate platform to the resin, followed by loading the drugs, and releasing the loaded platform from the solid support; and 10C showing examples of bound agents being activated with UV;

FIG. 11. is a reaction scheme showing the preparation of some platforms based on trihydroxybenzoic acid (THB); 11A showing creating a platform for binding nine molecules; 11B showing general THB structures with examples of reactive moieties and blocking groups for eventual binding two drugs, three molecules each; and 11C showing the construction of a dendrimer based on THB loaded with drugs;

FIG. 12. shows reactions useful in building the platforms; FIGS. 12A, 12B, 12C, and 12D show useful linkers and theirs use; and

FIG. 13. presents general formulae comprising some platforms of the invention; 13A shows Formula 13-1, FIG. 13B shows formulae from 13-2 to 13-8, including topological schemes, examples of attachment moieties, and examples of reactive groups for carrier binding; FIG. 13C lists some examples of reactive groups Y that may be included in the attachment moieties; and FIG. 13D presents examples of intermediate platform with reactive groups Y before activating them with the blocking groups P.

### DETAILED DESCRIPTION OF THE INVENTION

Multifunctional platforms have now been synthesized for coupling several agents, and for their subsequent differentiated release during the interaction with a biological environment. Two different drugs, for example, such as fludarabine and doxorubicine, coupled to the platform, were released each in a different manner when contacted with mouse serum or liver homogenate.

Multifunctional platforms, being actually dendrimer structures, are provided in the invention, for the attachment of multiple drugs and labels to any given carrier/transporter for targeted drug delivery. The known dendrimers, such as classical PAMAM (polyamidoamine) dendrimer or poly(propylene imine) dendrimer (see, e.g., Zeng, ibid.) comprise terminal groups of one type, all of which are equivalent from the viewpoint of chemical reactivity. The platforms of the invention comprise at least two terminal groups that differ by their nature and reactivity, and are suitable for loading various drugs, and further for attaching to a carrier, wherein the drugs are released sequentially, for example, after reaching their target.

The term carrier used throughout the description relates to a molecular structure to which a dendrimer is covalently linked, and which may assist in delivering a therapeutic or diagnostic agent to the desired site of action in the tissue, or near the treated tissue, wherein the assistance may include targeting said treated tissue or stabilizing said agent during its transport to the tissue. Said molecular structure may be a molecule or a part thereof or may be derived from such molecule, selected from protein, peptide, phospholipid, polysaccharide, biodegradable polymer, nucleic acid or a structural mimic thereof, such as a peptide nucleic acid (PNA); said molecular structure may be a molecule or a part thereof having high affinity to the treated tissue or its component, being e.g. a biopolymer or a small molecule; said molecular structure may be a molecule or a part thereof recognizing the targeted tissue or being recognized by the tissue, e.g. enzyme or antibody; said molecular structure may be a molecule or a part thereof that interacts with a regulation cascade in vivo, thereby initiating processes supporting the intended therapeutic goals; said molecular structure may be of biological or synthetic origin. The term therapeutic agent, or agent, is used to denote a molecular structure, or compound, covalently linkable to the platform of the invention, that, after being released from the platform, possibly truncated or enlarged during their cleavage from the platform, exhibits a benign effect when acting alone or together with other compounds, directly or by activating other compounds, wherein said benign effect may comprise damaging or neutralizing harmful molecules or microorganisms or cells, or said benign effects may comprise stimulating regulation cascades in the body involved in neutralizing harmful molecules or microorganisms or cells. The term diagnostic agent is used to denote a molecular structure, or compound, covalently linkable to the platform of the invention, that, after being released from the platform, possibly truncated or enlarged during their cleavage from the platform, participates in a diagnostic process. The term label is used throughout the description to denote a molecular structure that may assist in locating or visualizing the treated tissue, for example by being bound, noncovalently or covalently, to the treated tissue, or by being released near the treated tissue, which structure emits characteristic radiation by itself or when irradiated, or gives a detectable signal, which signal may be, for example chemical or electromagnetic.

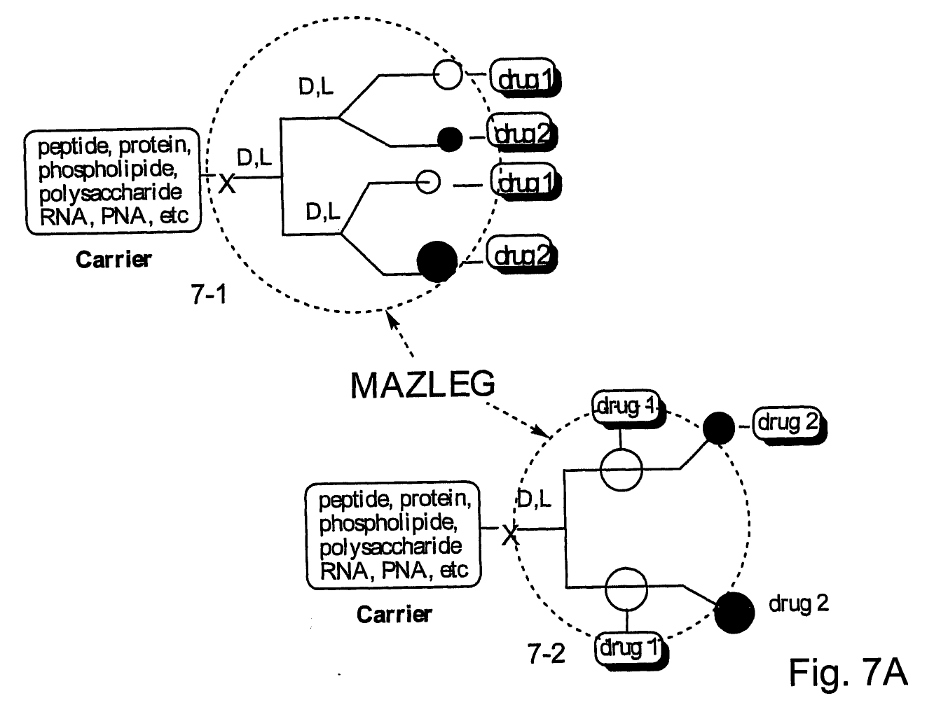

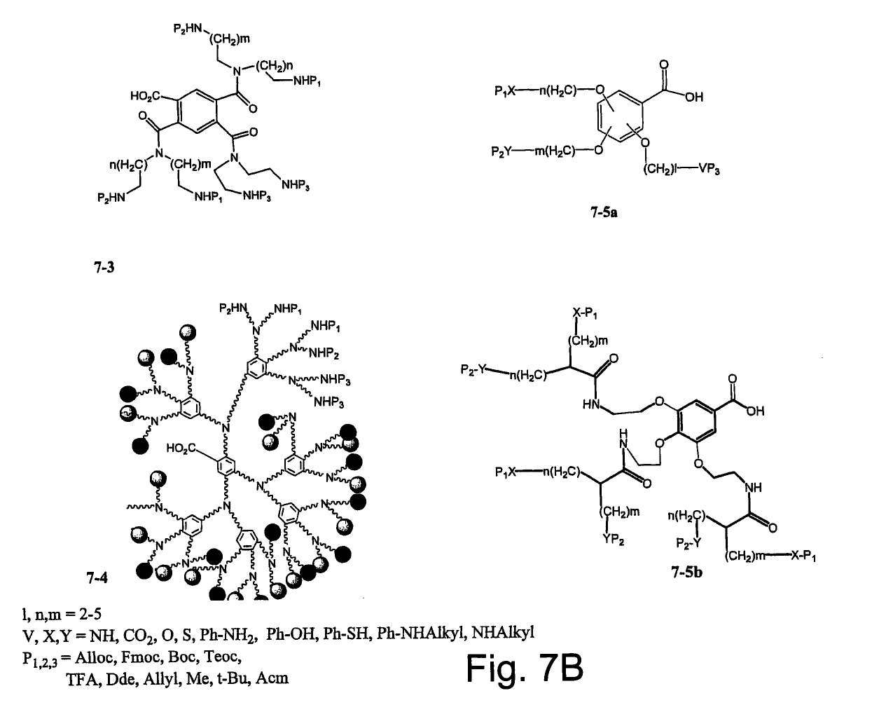

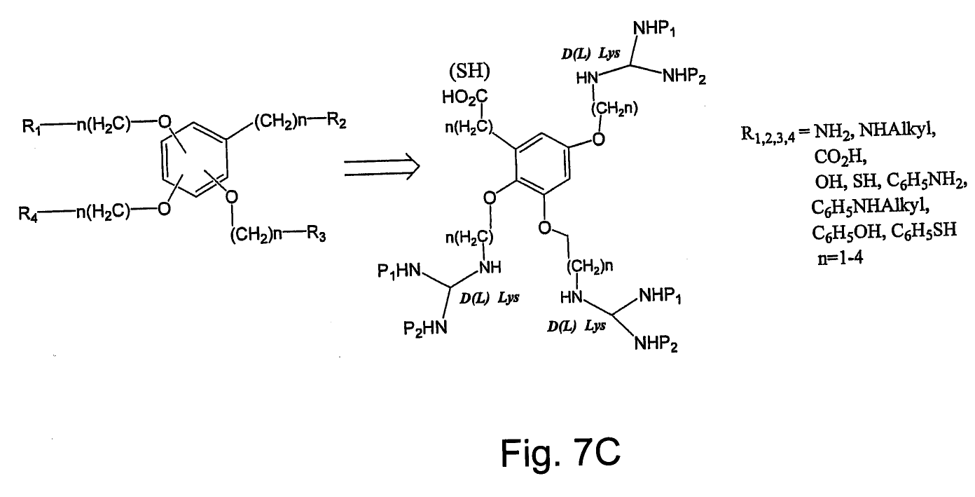

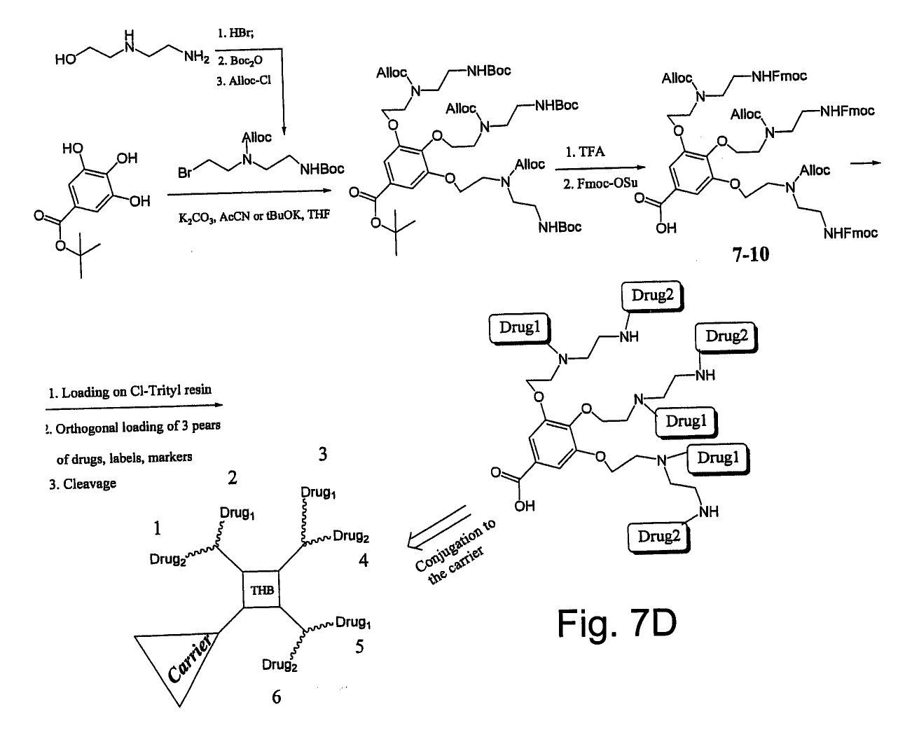

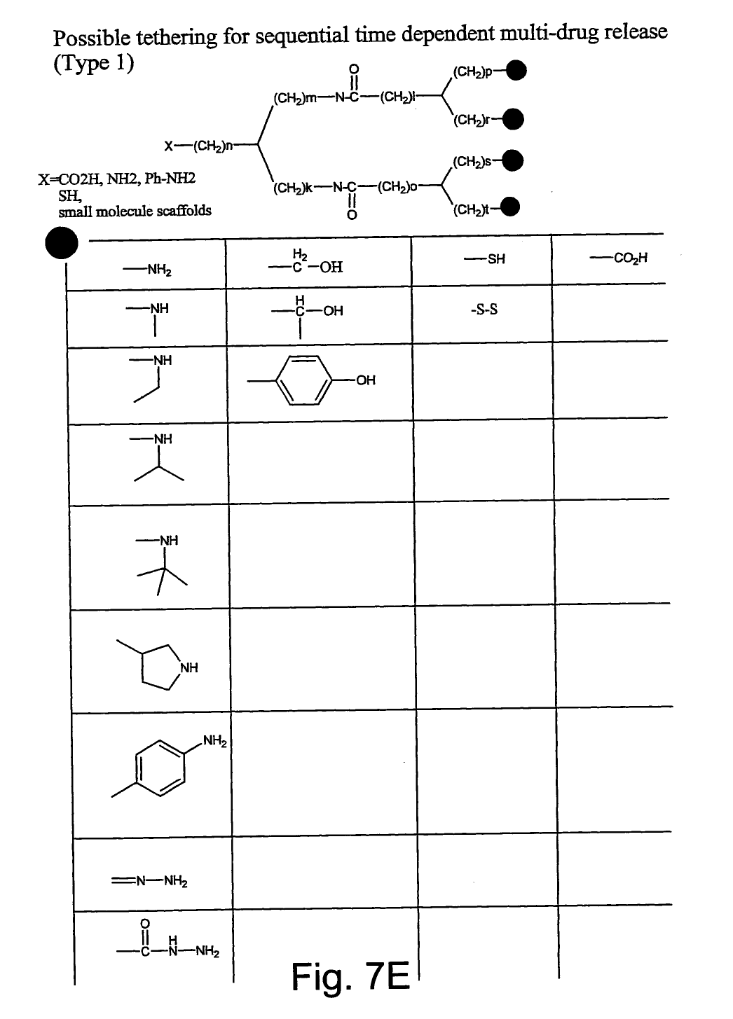

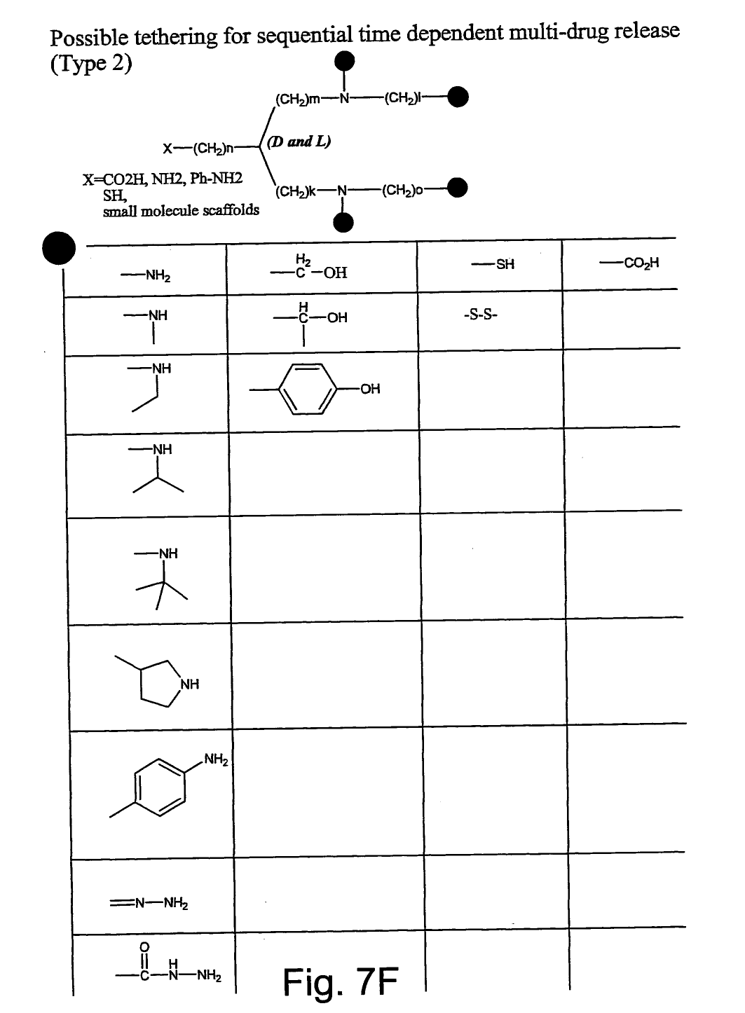

By way of illustration, two general platforms are presented in FIG. 7A, as general formulae 7-1 and 7-2. Full or empty circles in the figure represent terminal groups, i.e. the attachment points of the dendrimeric highest generation, which may be employed for coupling of a drug to be delivered, wherein the “drug” stands for any therapeutic agent or diagnostic label, alternatively the terminal groups may be blocking groups to be replaced by the drug molecules during the platform processing. The letters “D,L” at the branching points, i.e. points created by addition of a monomer unit to the terminal groups of a lower generation structure, indicate the sites of possible stereoisomerism. The loading capacity of the shown platforms is two molecules of drug 1 and two molecules of drug 2. The different properties of the two terminal groups result in two different molecular configurations of the coupled entities, even if the two coupled entities are the same molecules. This may be utilized in sequential release of a first part of the material at a time 1, and a second part of the same material at a time 2, depending on two different cleaving mechanisms. Alternatively, and more preferably in this invention, two different drugs, and that is what is illustrated in FIG. 7A, are coupled, each to different terminal group, and the platform, thus, enables to tune two separate release times for two different drugs, utilizing different functional attachments to the platform. For instance, in cancer therapy, two different drugs active in different anticancer mechanisms can be introduced into the body and released according to a required therapy regimen. In another preferred scenario, two different agents enter to the body, and by means of a carrier then enter to a cell in the targeted tissue, where two different intracellular mechanisms result in sequential cleaving and release of two materials at two different times, and possibly in two different subcellular compartments. In other possible scenarios, some of the agents may be released in serum, other in the cells, depending on the type of bond through which a drug is coupled to the platform, and depending on the availability of a relevant cleaving factor in the biological environment. Such factor may comprise, e.g., pH or the presence of hydrolytic enzymes.

Examples of a platform with free terminal groups, that can provide the above mentioned structures of 7-1 and 7-2, are shown, respectively, in FIG. 7E and FIG. 7F. The full circles represent radicals that may be selected independently from the tables in FIGS. 7E and 7F, respectively. A combined chemotherapy is indicated in various conditions, including proliferative diseases or infectious diseases, but the introduction of a plurality of different agents into the body brings specific problems comprising toxicities, drug interactions, etc. New delivery methods are needed that would improve specific targeting of active agents, thereby reducing unnecessary release of toxic compounds and their eventual undesired interactions.

The platform of the invention can be utilized for existing drugs and carriers, for example, by binding the platform, after loading it with two different known anti-tumor drugs, to a known receptor antibody, for use in a time-dependent, separate release of the drugs in the tumor cells having said receptor, thereby lowering the concentrations of the toxic drugs in blood/plasma/serum almost at zero value, if only the enzymes inside tumor cells will cleave said drugs. Optimal spatial and temporal distribution of a plurality of active agents, attainable by means of the invention, will reduce toxic effects of existing drugs, and would enable to introduce new agents, as well as to enhance the efficiency of existing therapies.

A platform according to the invention enables differentiated release of a plurality of drugs, of which tuning may include the order of their release, the time of their release, as well as the relative amounts of the released materials. A first drug, for example, may be coupled to a first type of the terminal groups of the dendrimer, creating a linkage configuration cleavable easily under the conditions of blood serum, whereas a second drug may be coupled to a second type of the terminal groups, creating a linkage configuration cleavable only by a protease existing in the target cell or in a subcellular compartment of the cell. The cleavage kinetics of both drugs will depend on the enzymes concentrations and activities, which may be well characterized, and on the structure of the dendrimer platform, which may be planned according to the needs. The mass ratio between the two drugs will depend on the ratio of the numbers of the two terminal groups, which depends on the type of monomer used in creating the dendrimeric platform of the invention and can be regulated.

The invention relates to a delivery means, multifunctional platform, for therapeutic and diagnostic agents, and the use of the platform according to the invention is limited only by the ability of said agents to be covalently coupled to the platform. The platform of the invention can be used for mixed chemotherapy, for photo dynamic therapy (PDT), for coupling PDT reagents and fluorescent materials, for radio labeling or for radio therapy, etc. Examples of active materials to be delivered include DNA chelating agents, tubuline metabolism inhibitors, fluorescent labels, and folic acid metabolism inhibitors.

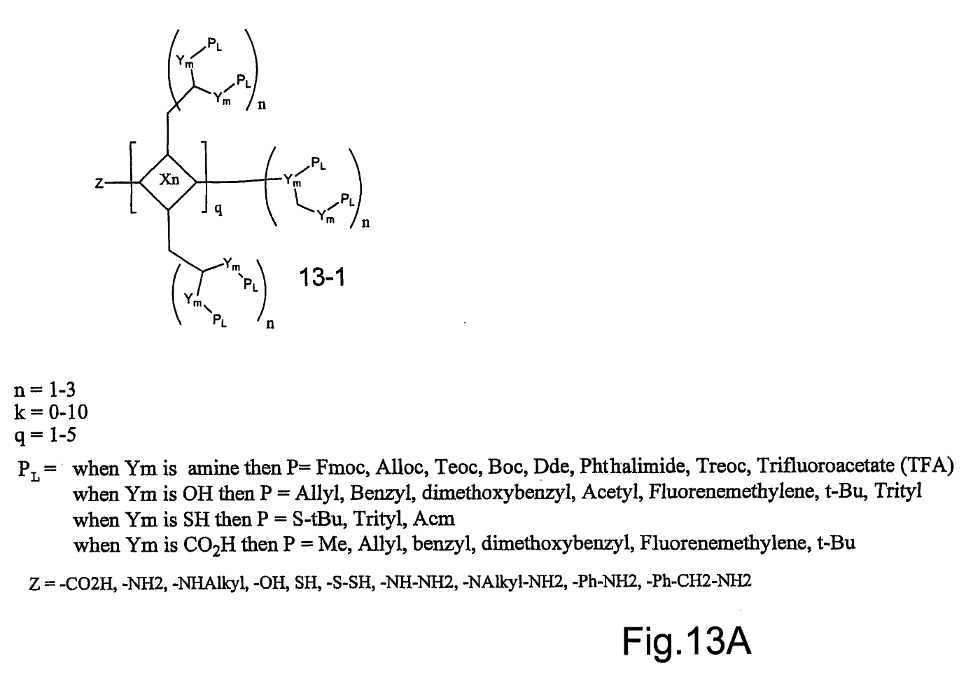

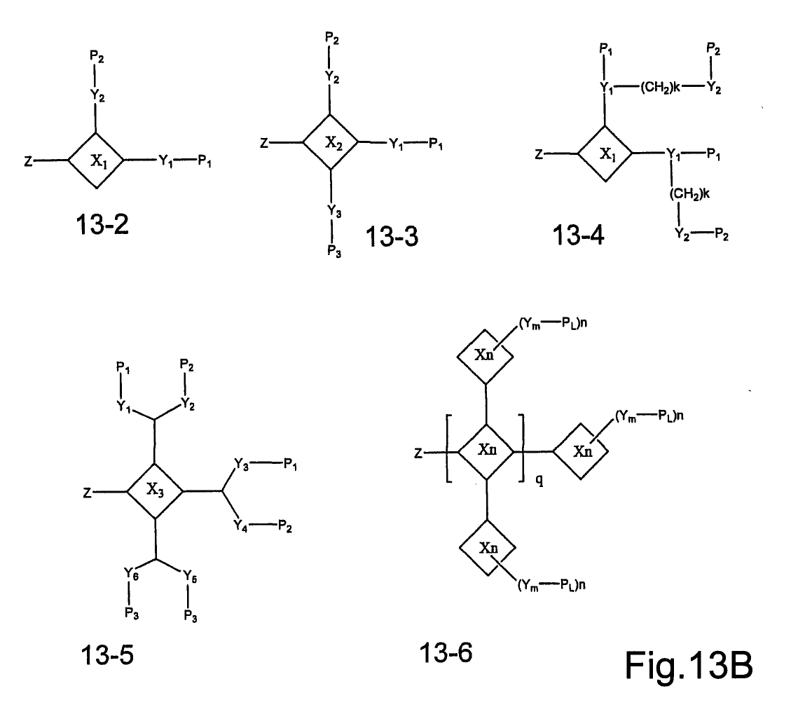

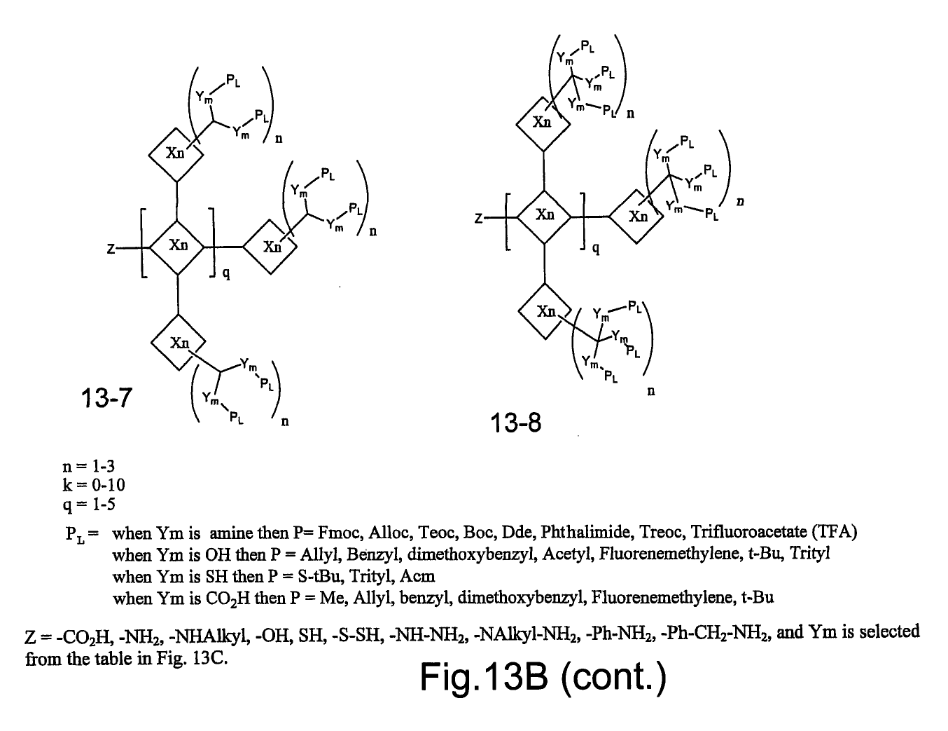

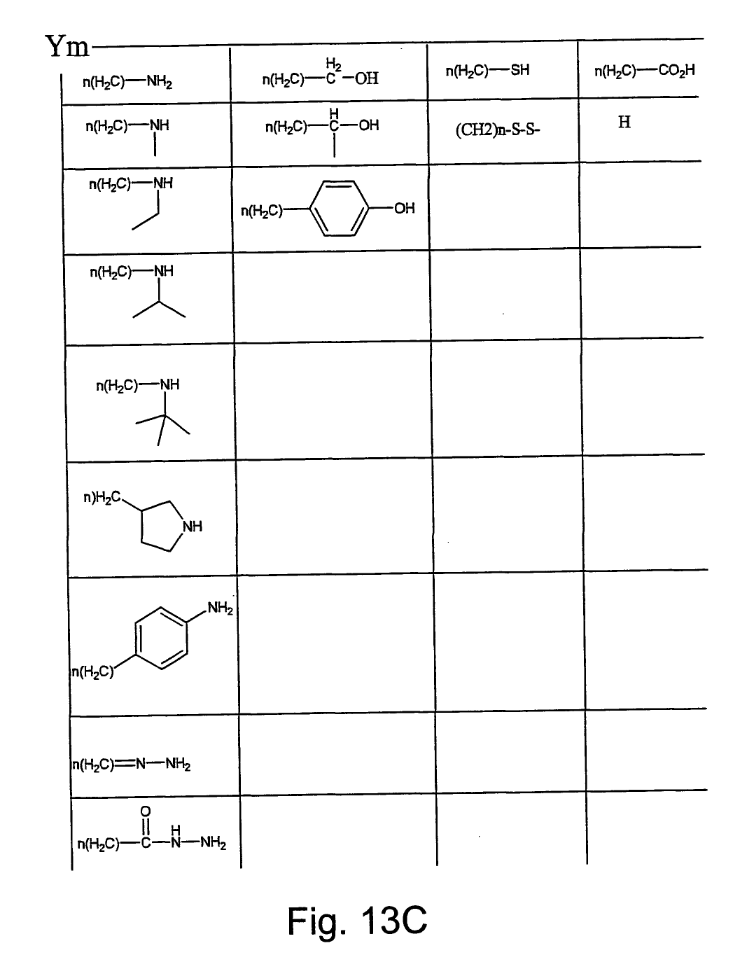

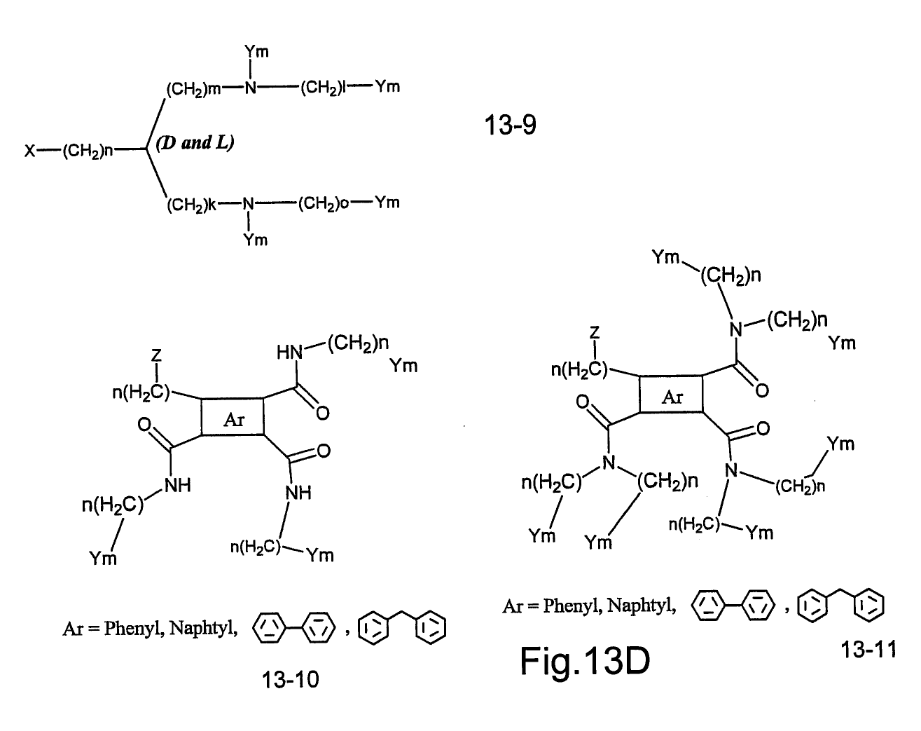

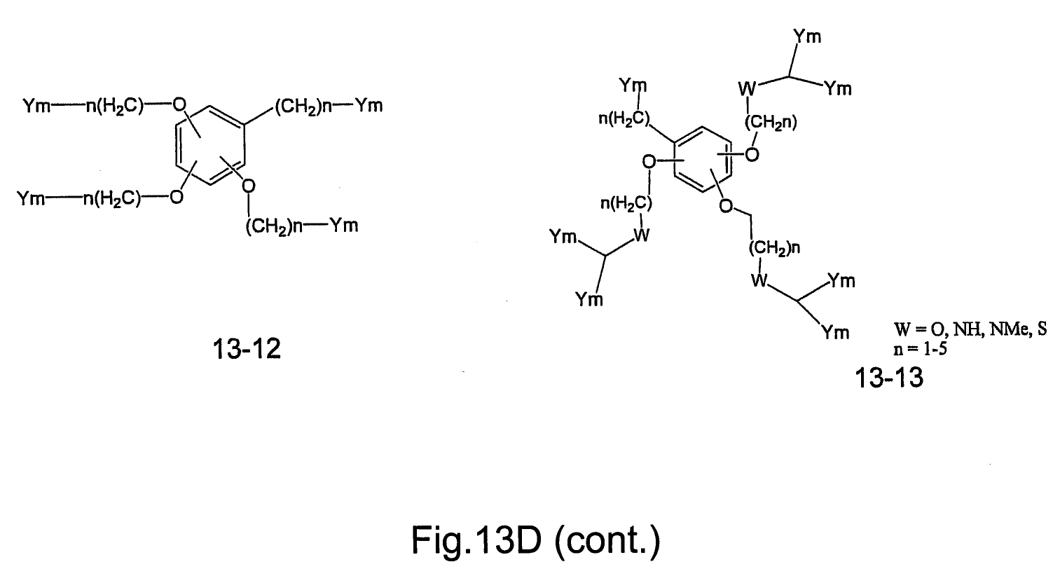

In a preferred embodiment of a multifunctional platform according to the invention, a dendrimeric platform is based on the general structure of Formula 7-5a as shown in FIG. 7B, comprising trihydroxybenzoic acid (THB) platform in the core, and various functional groups for coupling drugs, for example comprising linkers based on the groups presented in FIG. 13C.

In a preferred embodiment of a multifunctional platform according to the invention, a dendrimeric platform is based on the general structure of Formula 7-5b as shown in FIG. 7B, comprising THB platform in the core, and monomeric structures being amines or aminoacids, such as lysine. The structure 7-5 has two kinds of terminal groups, three groups of each kind.

In another embodiment, a dendrimeric platform is based on general the structure of Formula 7-3 as shown in FIG. 7B, based on pyromellitic dianhydride. The structure of Formula 7-3 has three kinds of terminal groups, two groups of each kind. Structure 7-4 is prepared from structure 7-3.

An empty, unloaded, dendrimeric platform of the invention can be synthesized from an intermediate bound to an immobilizing resin, to be consequently loaded with the drugs, and then cleaved off the resin and conjugated to a carrier. Another option, usable for example in the reactions illustrated in FIGS. 7C, 7D, is to prepare an empty platform in the solution, then to attach it to the resin and to load it with drug molecules, to cleave the loaded platform off the resin and to couple it to a carrier.

A multifunctional platform for delivery of at least two therapeutic or diagnostic agents according to the invention is based on a structure capable of forming at least three bonds, and may be selected from general structures schematically presented below as Formulae 13-1, 13-2, and up to 13-8 (see also FIGS. 13A and 13B).

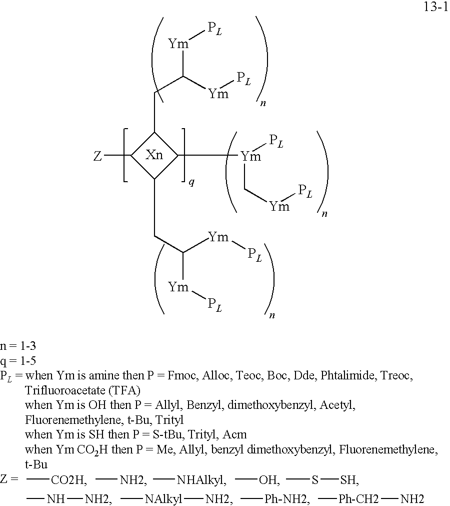

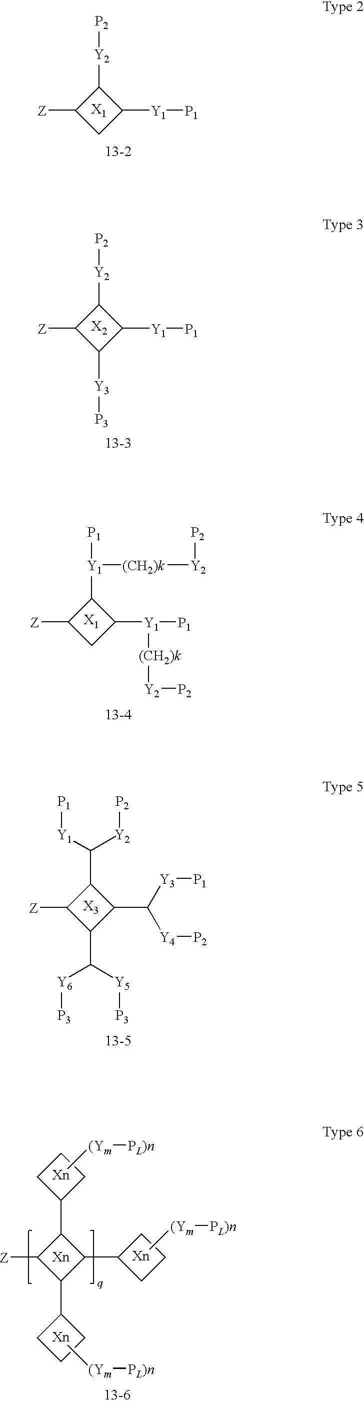

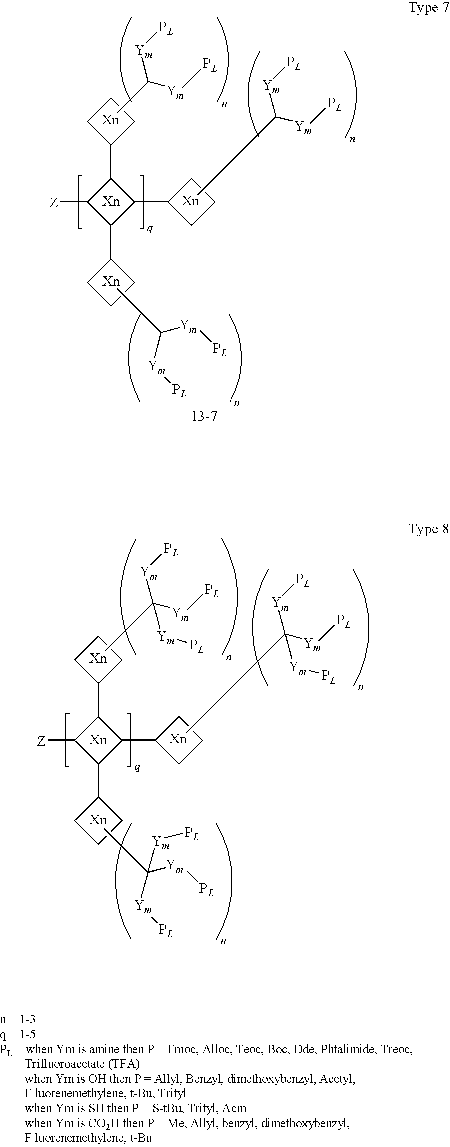

and wherein Ym are selected from the following structures (see also FIG. 13C):

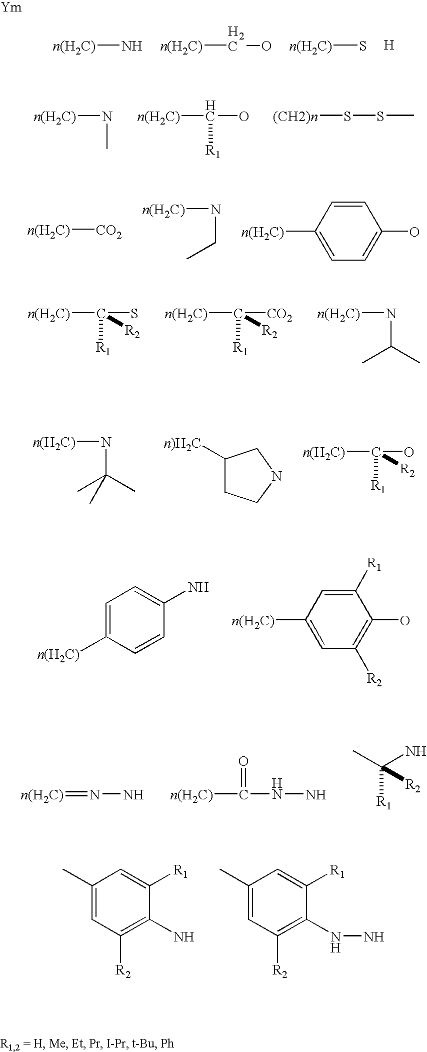

and wherein X are molecular structures capable of forming at least three covalent bonds, preferably X is a carbon atom, a cyclic structure—heterocyclic or aromatic. X may be, for example, selected from scaffolds of the following structures (see also FIG. 13D):

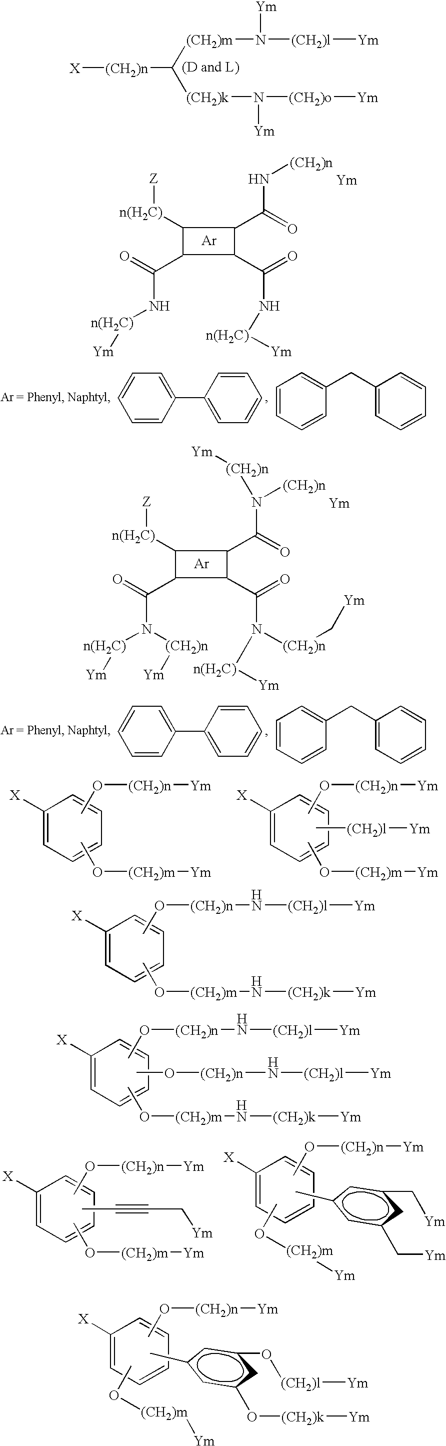

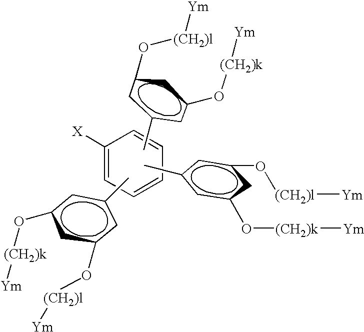

The above structures represent building blocks of the multifunctional platforms, when their terminal groups are reactive groups Yₘ, or they represent the activated platforms prepared for loading drugs, when their terminal groups are blocking groups P_{L}. In the above structures, Yₘ are independently selected from the above table, and two Yₘ groups in one structure may be different in this scheme.

In a preferred embodiment of the invention, a multifunctional platform of the invention for independent delivery of at least two drugs has a general structure described by Formula 14:

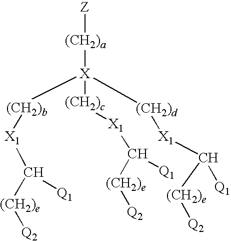

wherein X represents carbon atom, heterocyclic or aromatic ring selected from substituted benzene, naphthalene, diphenyl, phenylbenzyl;
Z is a reactive group selected from —COOH, —NH2, —NHalkyl, —OH, —SSH, SH, and —NHNH2;
a, b, c, d, and e are integers independently selected from 1 to 5;
X₁ is selected from —NH—, —NHCO—, and —CONH—, —O—, and —S—; and
Q₁ and Q₂ are groups independently selected from NHR, NHNR, COOR, OR, SR, S—SR, wherein R is selected from H, alkyl, aryl, and blocking groups, wherein said blocking group may be for example selected from Alloc, Fmoc, Boc, Teoc, TFA, and Dde, for NHR or NHNHR; from Acm, Trityl and s-tBu for SR or SSR, and from Me, Allyl, Benzyl, Dimethoxybenzyl, Fluorenemethylene for COOR, which blocking groups can be replaced by two different drug molecules, and wherein said reactive group Z couples said multifunctional platform to a carrier.

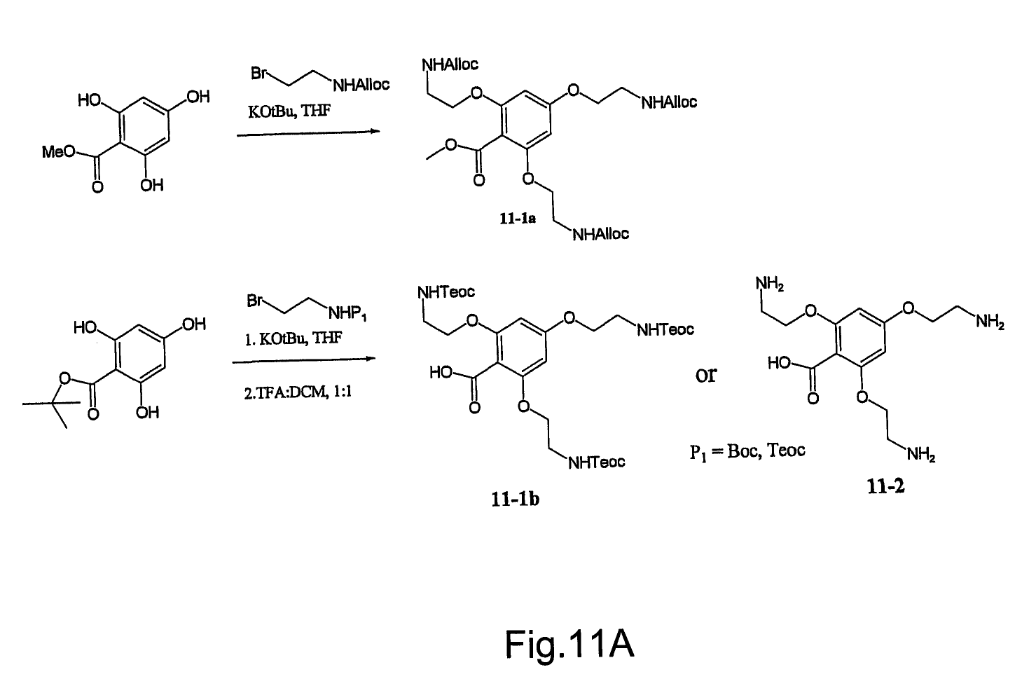

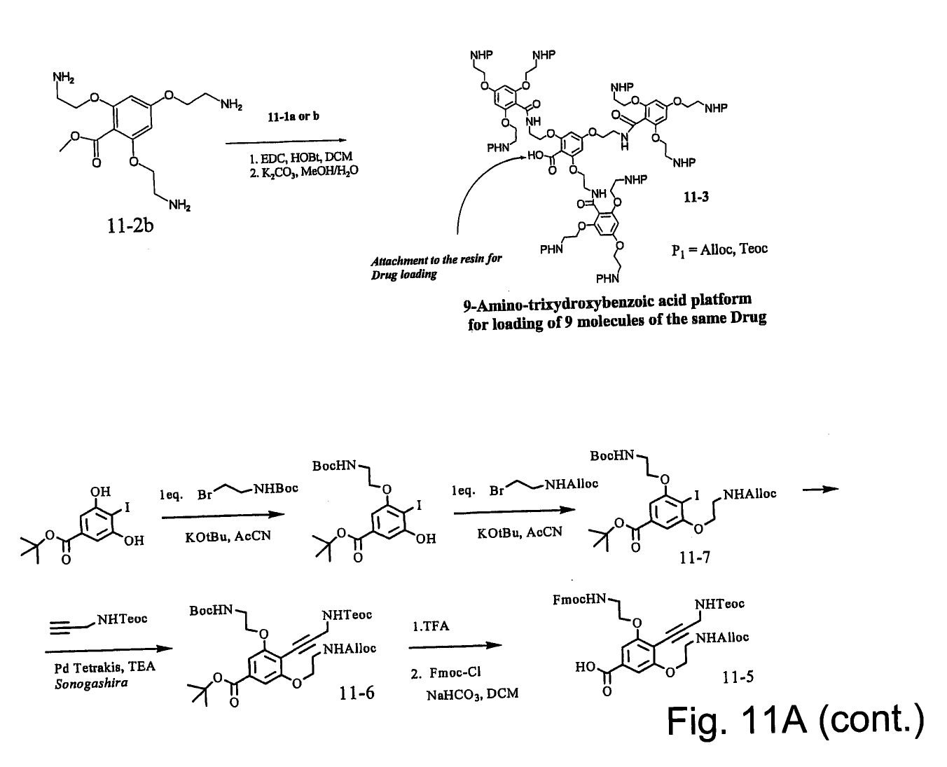

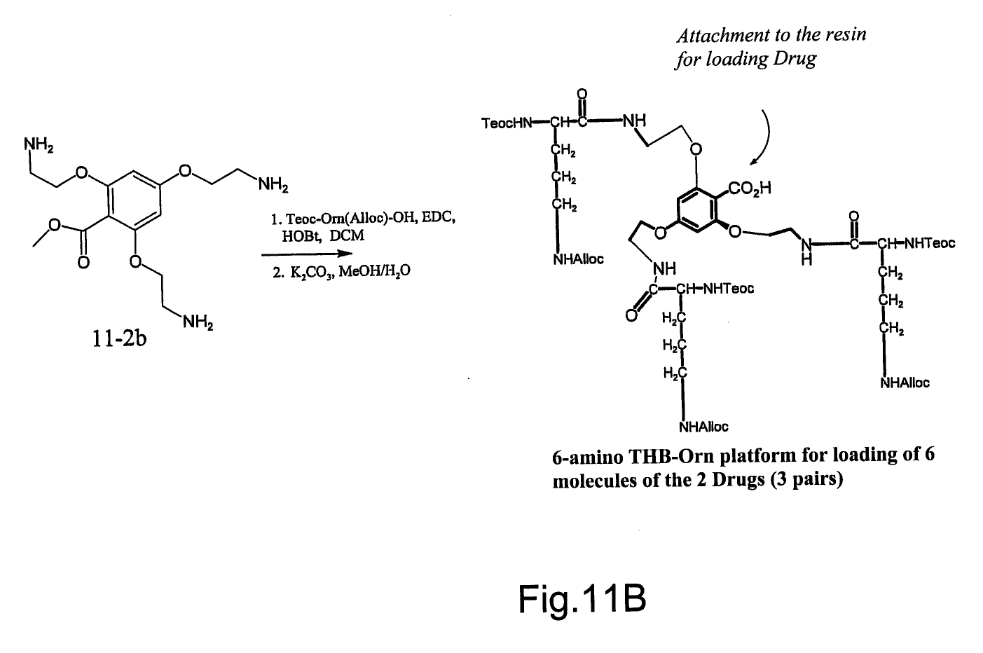

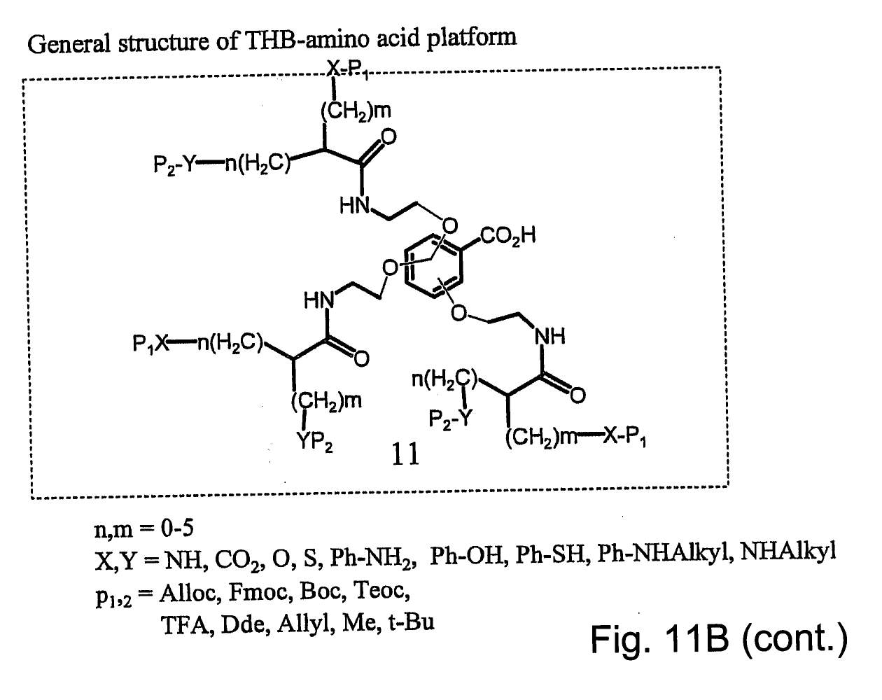

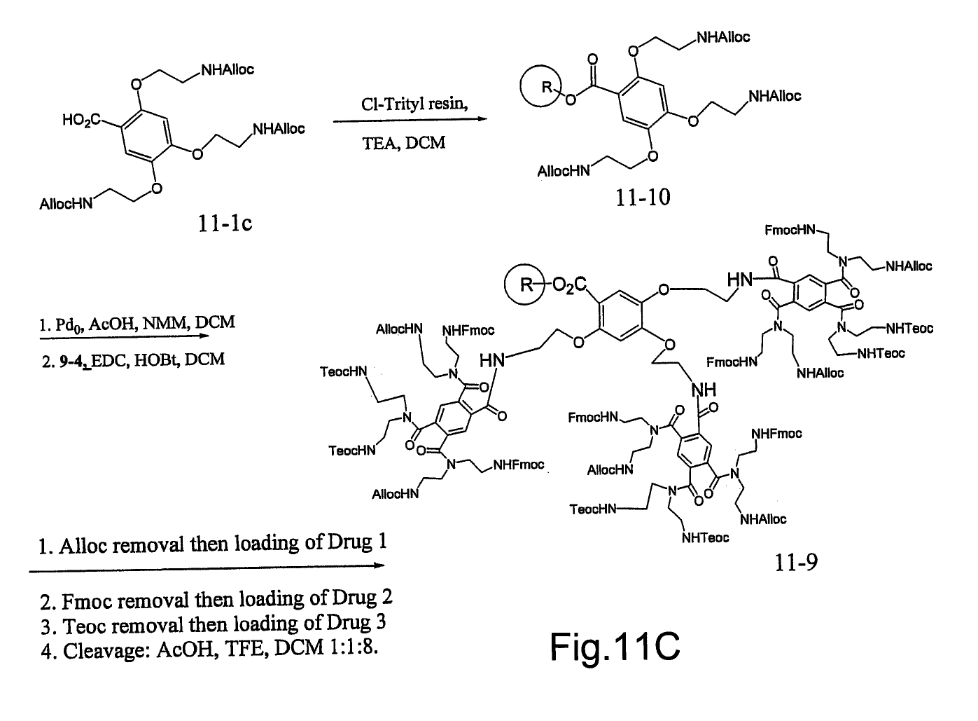

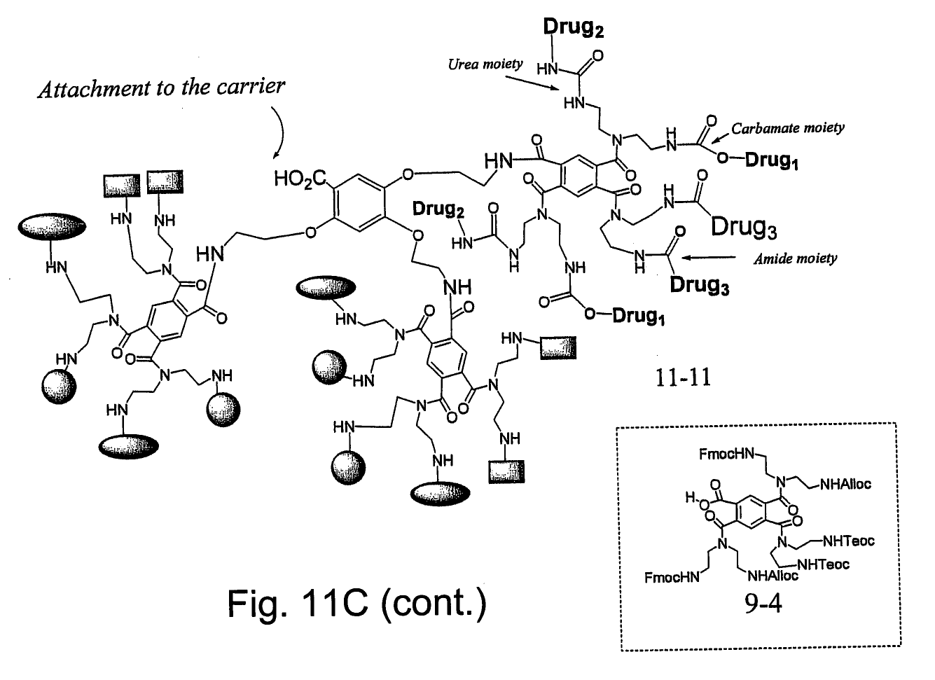

In a preferred embodiment of the invention, Z is —COOH. In another preferred embodiment X is trihydroxybenzoic acid (THB). Integers a and c may be for example 2, and integers b and d may be 4. The platform may have formula 11-8 as shown in FIG. 11B or formula 7-10 as shown in FIG. 7D.

The invention will be further described and illustrated in the following examples.

### EXAMPLES

### General

### Materials and Methods

HPLC solvents were H2O and CH3CN, both containing 0.1% (v/v) TFA. For analytical HPLC, a Cosmosil 5C18-AR column (4.6 250′ mm) was eluted with a linear gradient of CH3CN at a flow rate of 1 mL/min on a Waters™ 717 plus autosampler equipped with a Hitachi D-2500 chromatointegrator. Preparative HPLC was performed on a Waters Delta Prep 4000 equipped with a Cosmosil 5C18-AR column (20 250′ mm.) using a linear gradient of CH3CN at a flow rate of 15 mL/min. Ionspray (IS)-mass spectra were obtained with a Sciex APIIIIE triple quadrupole mass spectrometer (Bar-Ilan Un., Israel). Protected amino acids, CL-trityl resin amide resin and other chemicals were purchased from Sigma-Aldrich.

### Fludarabine and Doxorubicine Conjugates Cleaved by Mouse Serum

Behavior of Fludarabine & Doxorubicine conjugates were checked in vitro in mouse serum. Test compounds (100 nmol) were dissolved in mouse serum (100 μL)—H₂O (100 μL), and incubated at 37° C. At intervals, an aliquot was sampled and examined by analytical HPLC with a linear gradient of CH3CN (10-40%, 30 min). HPLC peaks of the starting compound and the generated products were identified by IS-MS analysis. The amounts of the starting compound and the generated products were quantitated from the corresponding peak areas.

### Fludarabine and Doxorubicine Conjugates Cleaved by Mouse Liver Homogenate

Behavior of Fludarabine & Doxorubicine conjugates were checked in vitro when contacted with mouse homogenate. mouse liver (21.4 g) was suspended in ice-cold PBS (85 mL) and then homogenized, followed by centrifugation at 3000 rpm for 10 min. The obtained supernatant was diluted to 40% (v/v) solution with PBS. Test compounds (100 nmol) were dissolved in PBS (100 μL), which contained 0.1% (v/v) m-cresol as an internal standard. After addition of 40% (v/v) mouse liver homogenate solution (100 μL), the mixture was incubated at 37° C. At intervals (0, 1, 2, 4, 6, 10 and 24 h), a 10 μL aliquot was sampled. After quenching enzymatic activities by addition of 0.1 M aq. HCl (190 μL), 6 M guanidine-HCl-1 M Tris buffer (pH 7.5, 300 μL) was added and the mixture was then stirred for 12 h. 100 μL of this solution was analyzed by analytical HPLC with a linear gradient of CH3CN (10-40%, 30 min). HPLC peaks of the starting compound and the generated compounds were identified by IS-MS analysis. Their amounts were quantitated from the corresponding peak areas, which were corrected by the internal standard m-cresol.

### Example 1

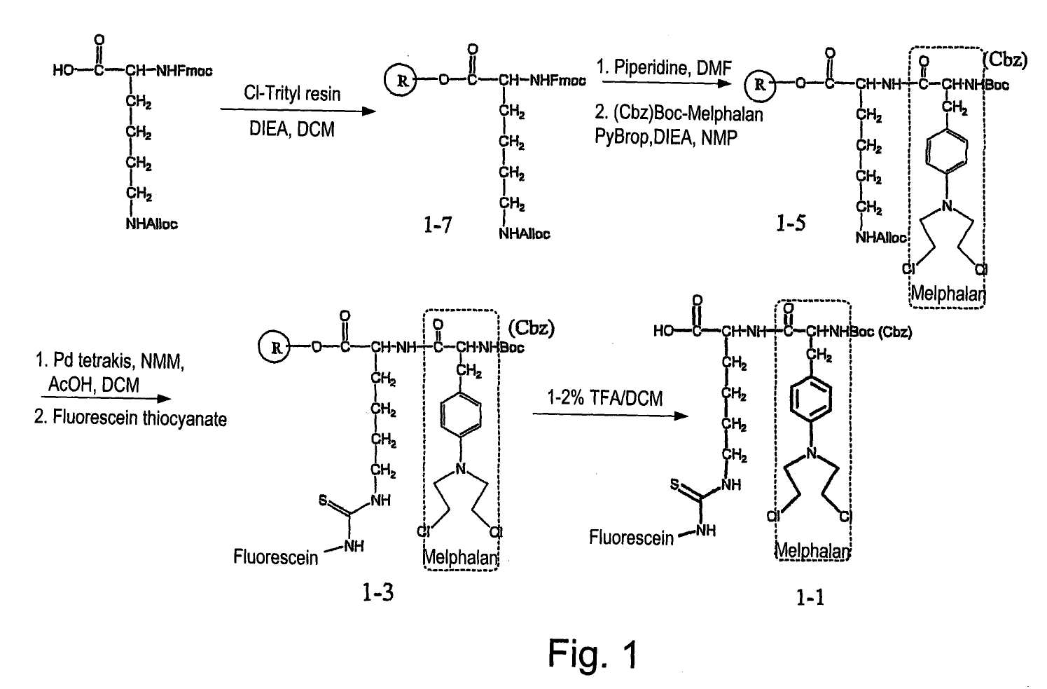

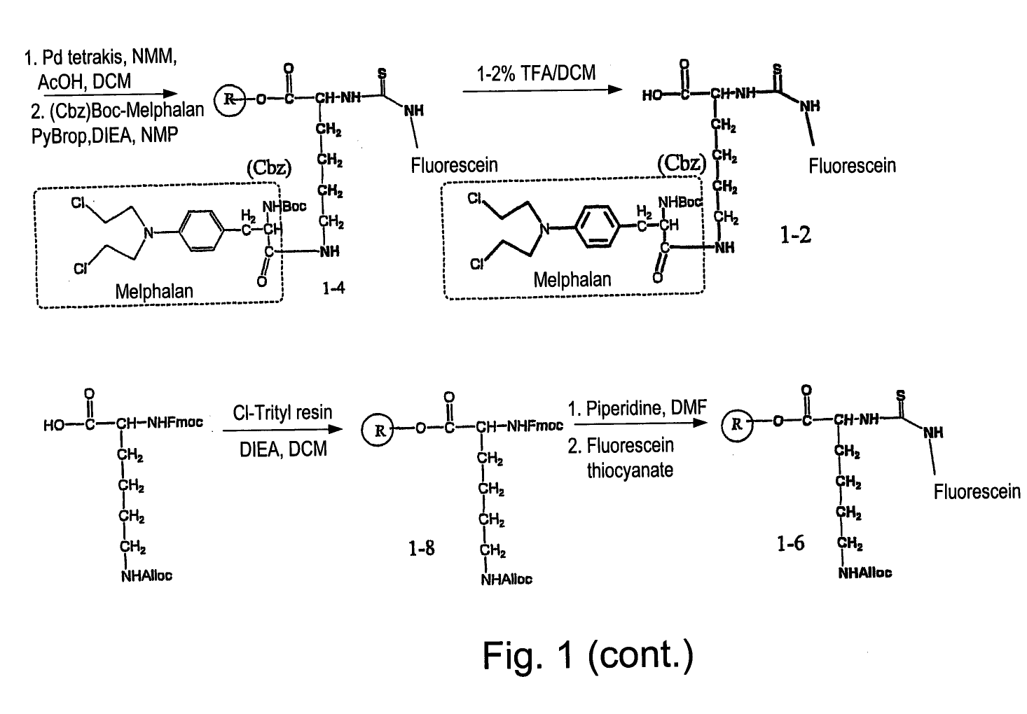

The release from a bifunctional platform of the invention was compared for two “drugs”, namely melphalan and fluorescein, in a biological environment, simulated in vitro contacting the conjugates with mouse liver homogenate. The two compounds were coupled to the platform via two different types of chemical bonding, via amide bond and via thiourea bond, also varying the linker length. Fluorescein is coupled through the 6-amine and melphalan through the α-amine. FIG. 1 shows the experimental details. The release of the two drugs from the platform to which they were bound by two different bonds was monitored by LC-MS, using standards of Lys, melphalane, fluorescein-derivative.

### Example 2

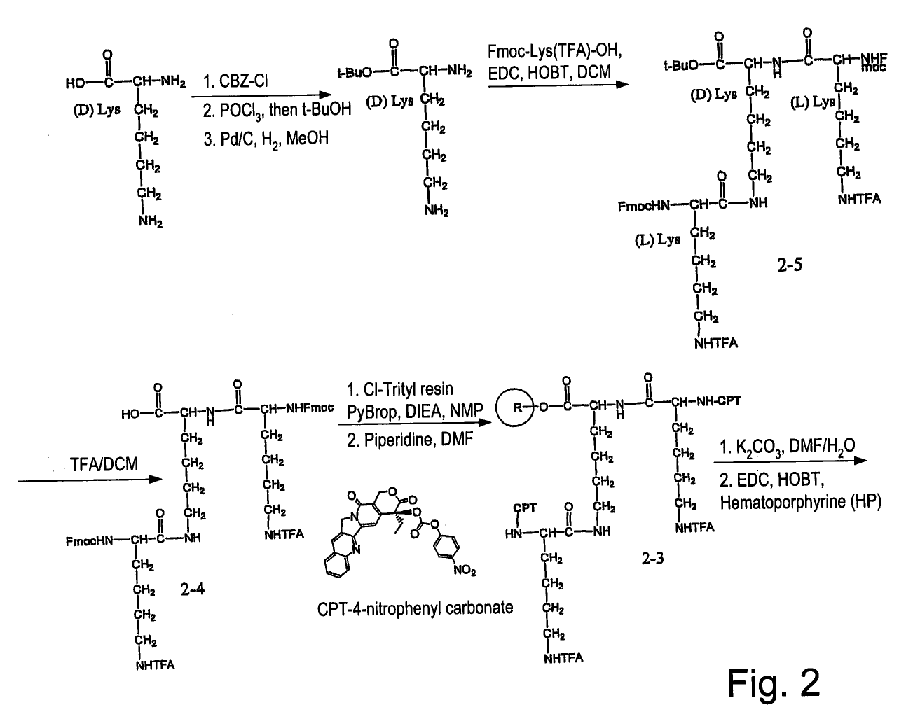

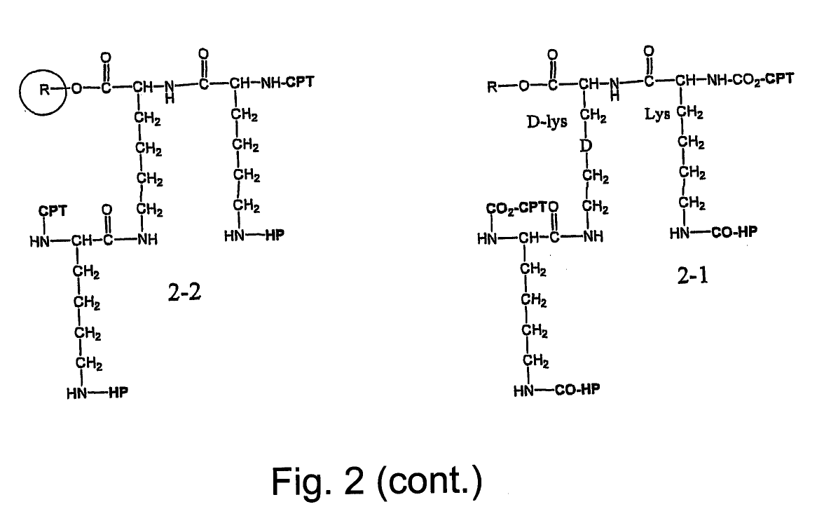

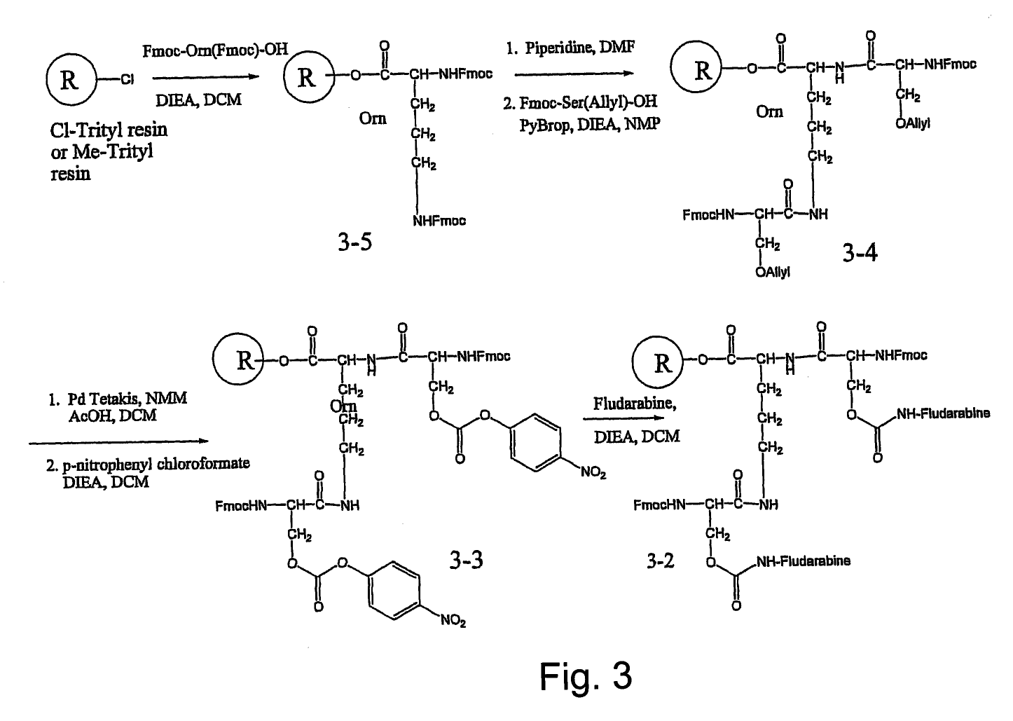

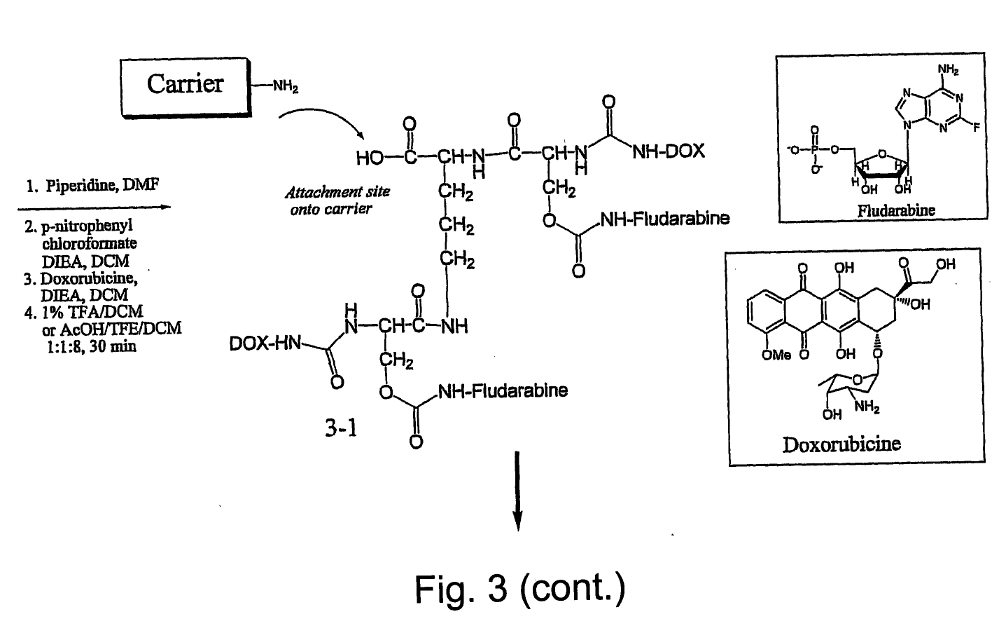

A tetra-valent platform was prepared, and was conjugated with two drugs, camptothecin and hematoporphyrin, and then the release rates from the platform were checked in vitro. Different linker lengths and chemical characters were employed, and mouse liver homogenate, as a source of enzymes for cleaving the drugs off the platform was used. The cleavage was monitored by LC-MS using the standards of Lys, D-Lys-Lys, camptothecin, and hematoporphyrin. The word drug is used in the Examples in the sense outlined in the description, meaning compounds used for therapy or diagnosis, as well as model compounds characterizing the used system. Hematoporphyrin, for example, may be useful in photodynamic therapy (PDT). FIG. 2 shows additional details of the experiment.

The protected peptidyl resin was manually constructed using Fmoc-based solid-phase synthesis [see, e.g., Hirokazu T.: Org. Biomol. Chem. 1 (2003) 3656-62] on an Cl-trityl resin (0.64 mmol/g, 0.1 mmol scale, (D)-Lys-Fmoc Lys(TFA)-OH amino acid (2.5 equiv.) were successively condensed using DIEA (Diisopropylethylamine) (7.5 equiv.) in DCM. The Fmoc-group was deprotected by treatment of the resin with 20% (v/v) piperidine-DMF for 1 and 15 min. TFA group was deprotected using K2CO3 in DMF/Water. The camphocethine was loaded onto the platform by preparing its p-nitrophenol carbamate (p-nitrophenyl chlorophormate, DCM, TEA) and coupling to D-Lys-Lys in DCM, DIEA. Hematoporphyrine was coupled by usual procedure (EDC, HOBt, DCM). The resulting protected peptidyl resin (50 μmol) was treated with 1% TFA (5 mL) or AcOH, trifluoroethanol in DCM (1:1:8) in the presence 1,2-ethanedithiol (100 μL, 33 equiv.) for 30 min. After removal of the resin by filtration, the filtrate was concentrated in vacuo. Ice-cold dry diethyl ether (30 mL) was added to the residue. The resulting powder was collected by centrifugation and then washed three times with ice-cold dry diethyl ether (20 mL) obtaining the crude compound The crude product in the solution (AcOH/H2O 1:1) was purified by preparative HPLC to afford a pure compound. The purity was determined by analytical HPLC. The structures were confirmed by 1H NMR and LC-MS.

### Example 3

A tetra-functional platform was prepared and conjugated with acid sensitive fludarabine and doxorubicine, and their release rates were compared in vitro with mouse plasma and liver homogenate, as described above. This example encompasses the technology for loading of the acid sensitive drugs such as DOX and Fludarabine like molecules (Arabinoside, Gemcitabine, Cladribine) onto Orn-Ser based platform. The raw platform is built on Cl-Trityl resin and, after loading with the drugs, is cleaved from the resin under very mild conditions in a free carboxyl composition. The platform may be linked to a carrier through the amine bond (through side amine chain of Lys in antibody, enzyme, peptide or any other amine containing carrier). The cleavage from the resin conditions are: 1% TFA/DCM, 15 min or AcOH/Trifluoroetanol (TFE)/DCM, 30 min in 1:1:8 ratio.

The protected peptidyl resin was manually constructed using Fmoc-based solid-phase synthesis [see, e.g., Hirokazu T.: Org. Biomol. Chem. 1 (2003) 3656-62] on acid super sensitive Cl-Trityl resin (0.64 mmol/g, 0.1 mmol scale. Fmoc Orn(Fmoc)-OH amino acid (2.5 equiv.) were successively coupled using DIEA (2.5 equiv.) (Diisopropylethylamine) (7.5 equiv.) in DCM. The Fmoc-group was deprotected by treatment of the resin with 20% (v/v) piperidine-DMF and next Fmoc-Ser(Allyl)-OH was coupled using PyBrop, DIEA, NMP. The Allyl group of protected platform was deprotected using Pd Tetrakis, AcOH, NMM in DCM, followed by the activation with p-nitrophenyl chloroformate, DIEA, DMAP, DCM, using the standard procedure. The first drug (fludarabine) was coupled in DCM in presence of DIEA, DMAP. The Fmoc-group was deprotected by treatment of the resin with 20% (v/v) piperidine-DMF for 1 and 15 min. The p-nitrophenyl chloroformate was reacted with the free amino group in DCM with DIEA to form activated carbamate. The second drug (doxorubicine) was coupled (DIEA on DCM). The resulting protected peptidyl resin (50 μmol) was treated with 1% TFA in DCM at 4° C. for 15 min. or alternatively with AcOH/Trifruoroethanol or Hexafluoroisopropanol/DCM in ratio 1:1:8. for 30 min. After filtration the resulting mixture was evaporated and then washed three times with ice-cold dry diethyl ether (20 mL) affording the crude compound The crude product was purified by preparative HPLC to yield a pure compound. The purity was determined by analytical HPLC. The structures were confirmed by 1H NMR and LC-MS.

### Example 4

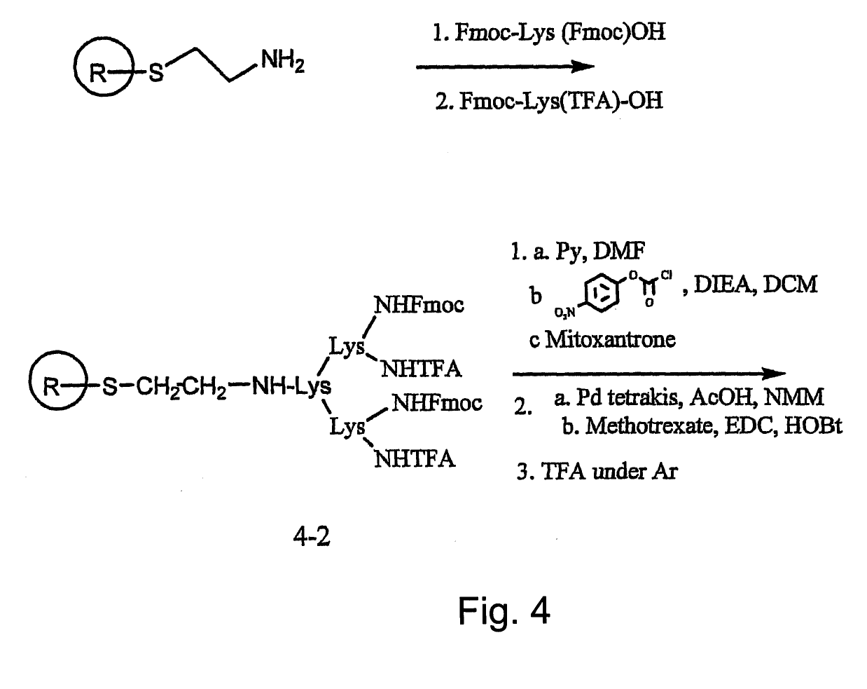

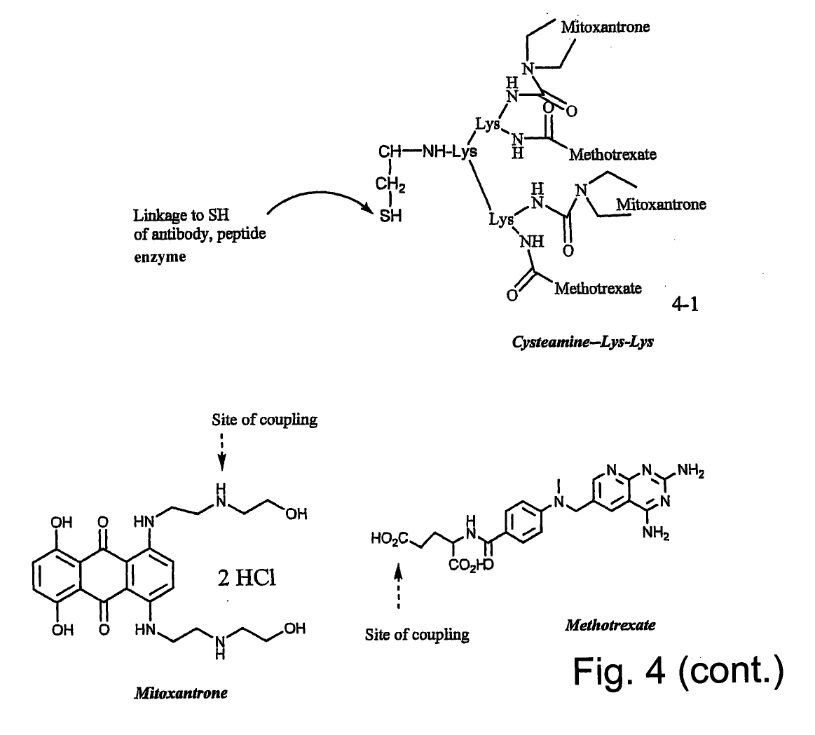

A tetra-functional platform was prepared by the known methods of solid phase organic chemistry (SPOC), and was conjugated with mitoxantrone and mithotextrate, employing Cysteamine as —SH linker, and the release rates were compared in vitro as described above. Additional details are in FIG. 4. Four drug molecules were loaded per platform (2 mitoxantrone and 2 mithotextrate). The platform has a —SH linker (Cysteamine) for coupling with carriers through S—S bond formation. The platform was built on Cl-Trityl resin preloaded with cysteamine and after loading the drugs is cleaved from the resin by TFA under Argon in free SH Formula. The Drugs were coupled to the platform by secondary amide (methotrexate) and urea moiety (mitoxantrone).

The protected peptidyl resin was manually constructed using Fmoc-based solid-phase synthesis on Cl-Trityl resin preloaded with cysteamine (0.60 mmol/g, 0.1 mmol scale. Fmoc Lys (Fmoc)-OH amino acid (2.5 equiv.) were successively coupled using PyBrop (2.5 equiv.), DIEA (disopropylethylamine) (7.5 equiv.) in NMP. The Fmoc-group was deprotected by treatment of the resin with 20% (v/v) piperidine-DMF and next Fmoc-Lys(Allyl)-OH was coupled using PyBrop, DIEA, NMP in the same manner. The Fmoc-group was deprotected by treatment of the resin with 20% (v/v) piperidine-DMF for 1 and 15 min. p-Nitrophenyl chloroformate was reacted with the free amino group in DCM with DIEA to form activated carbamate. The first drug (mitoxantrone) is coupled in DCM in presence of DIEA. The allyl group of protected platform was deprotected using Pd Tetrakis, AcOH, NMM in DCM. The second drug (methotrexate) is coupled (EDC, HOBt, DCM). The resulting protected peptidyl resin (50 μmol) was treated with 95% TFA (degassed), 2.5% H2O and 2.5% TIS for 30, min under argon. After filtration, the resulting mixture was evaporated and then washed three times with ice-cold dry diethyl ether (20 mL) affording the crude compound. The crude product was purified by preparative HPLC to yield the pure compound. The purity was determined by analytical HPLC. The structures were confirmed by 1H NMR and LC-MS.

### Example 5

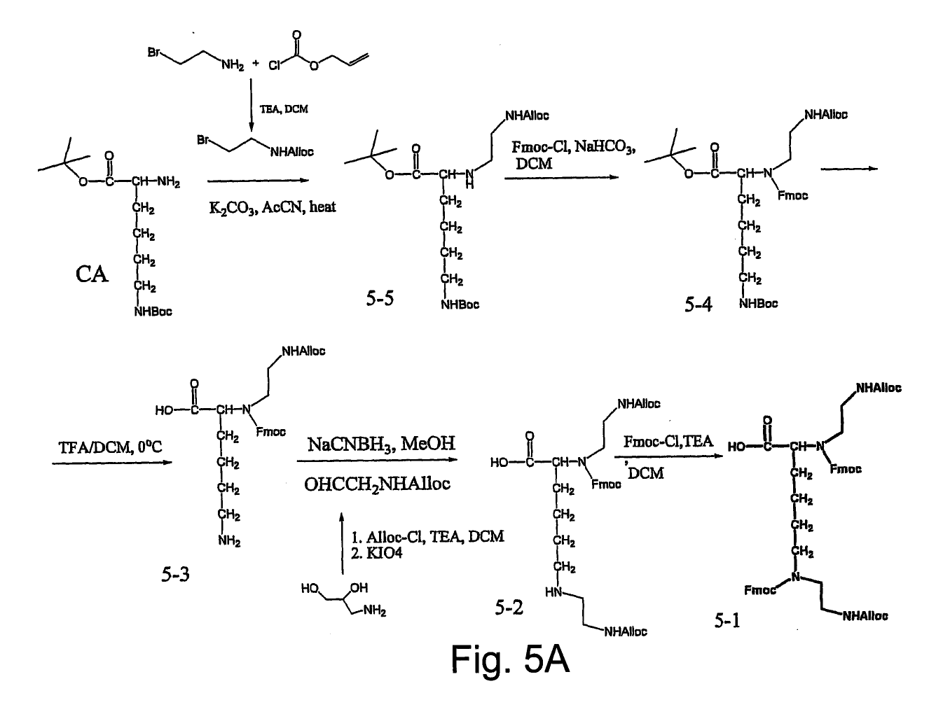

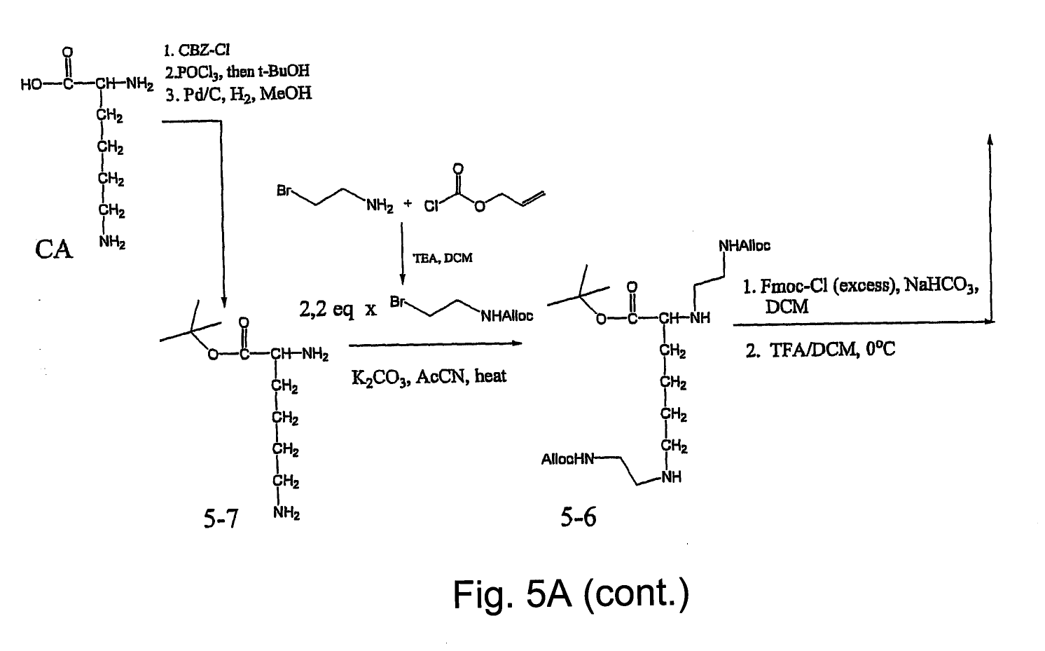

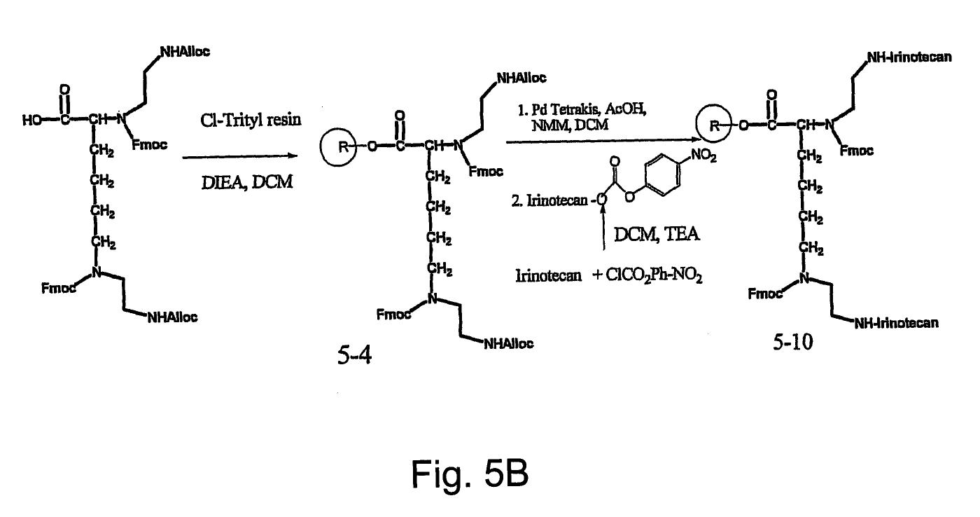

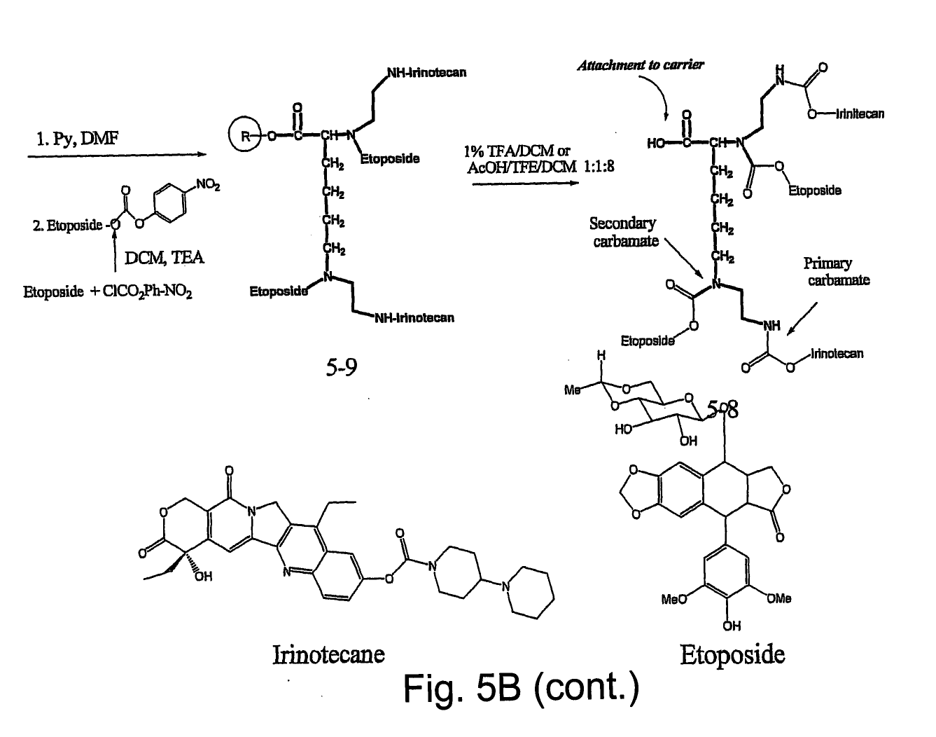

A tetravalent platform according to the invention was prepared, for loading two different drugs, two molecules each, by SPOC, and the comparison of their release rates in vitro was performed. The tetra-functional platform was prepared in solution, four drug molecules per platform were loaded (2 irinitecane and 2 etoposide) by SPOC. FIG. 5A shows details. The Fmoc, Alloc protected platform was prepared from Lys tBu ester [Gellerman G. et al.: J. Pep. Res. 57 (2001) 277] by regiospecific double alkylation (pathway 5-6 in FIG. 5A) with 2.2 eq. of Alloc amino alkyl bromide [Segheraert C.: J. Chem. Soc., Perkin Trans. 1, 6 (1986) 1061-4] followed by double Fmocilation and hydrolysis (TFA). Another way is longer and starts from commercially available Lys(Boc)-O-tBu (see pathway 5-5).

The loading of the drugs is performed on an acid super sensitive solid support (Cl-Trityl resin) ending as a carboxylic acid ready for conjugation with carrier. After loading on the resin (5-1, DIEA, DCM), Alloc was removed and preactivated. Irinotecan was coupled to form 5-10 in FIG. 5B (p-nitrophenyl-CO2Cl, TEA, DMAP, DCM).

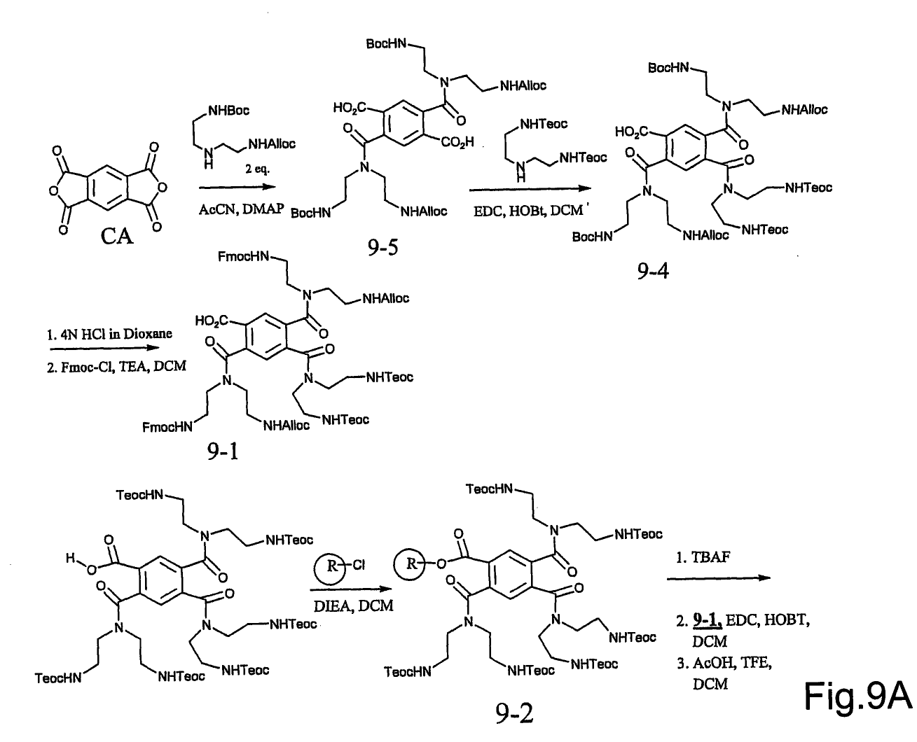

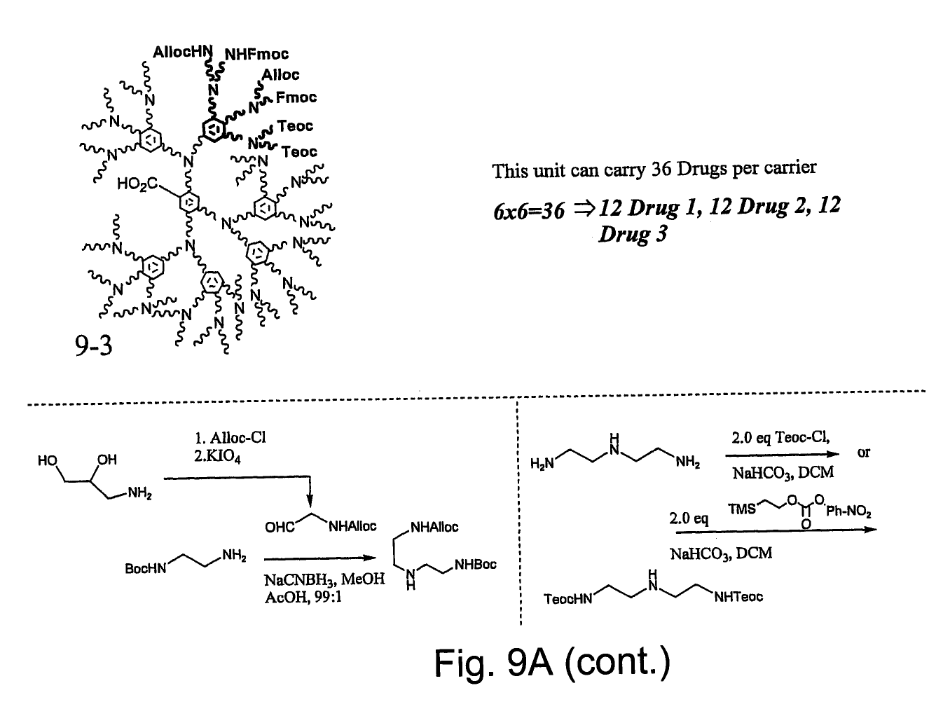

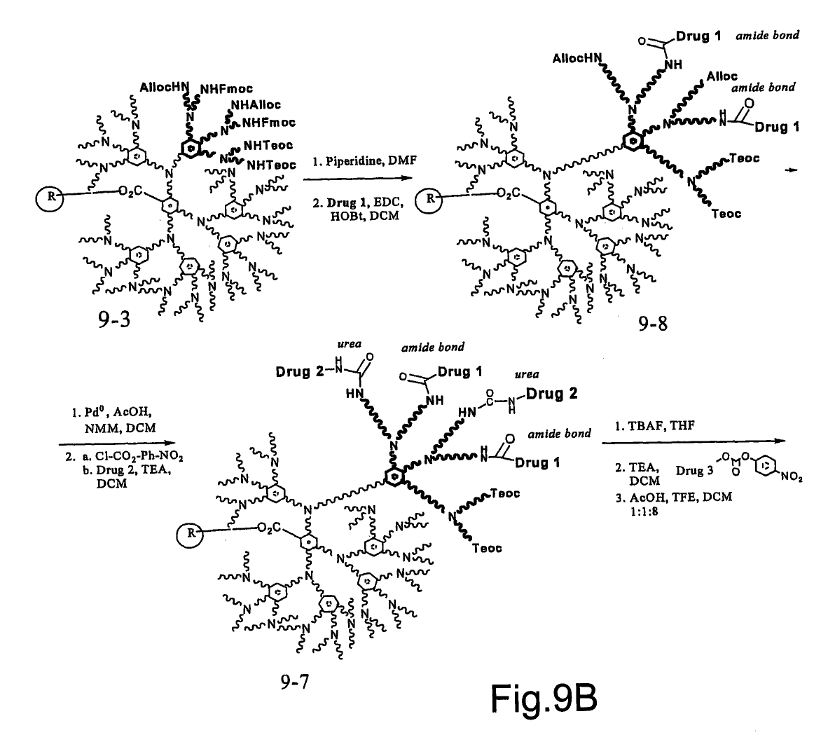

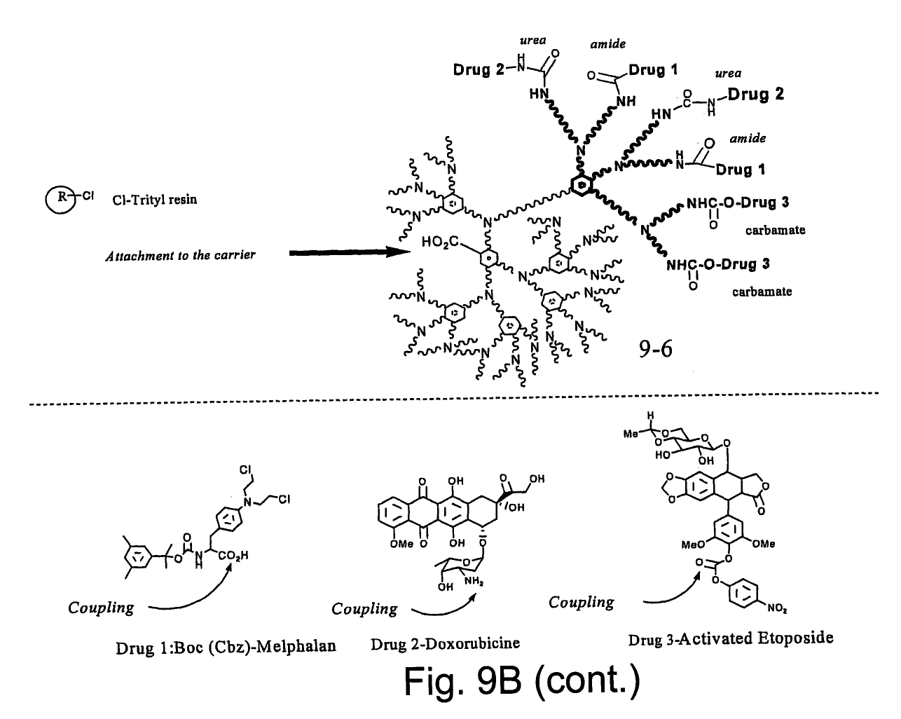

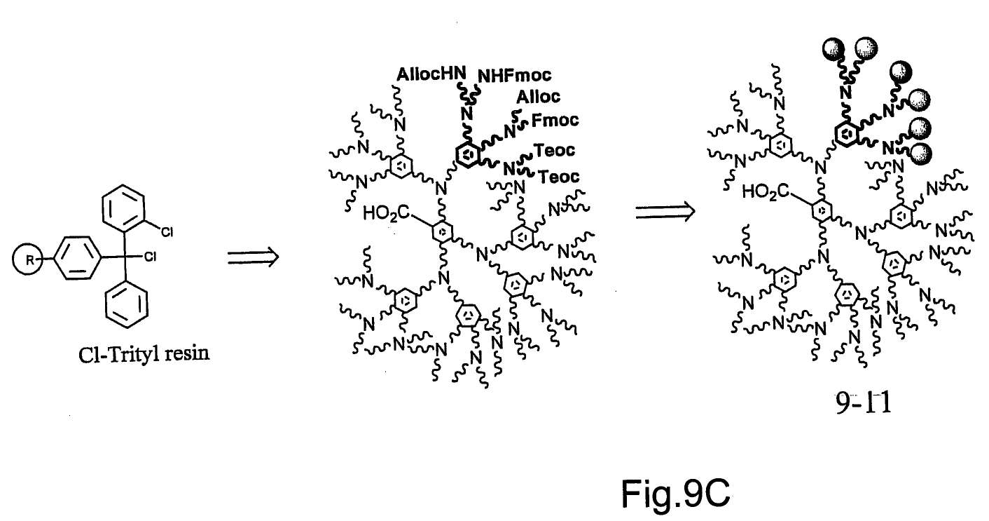

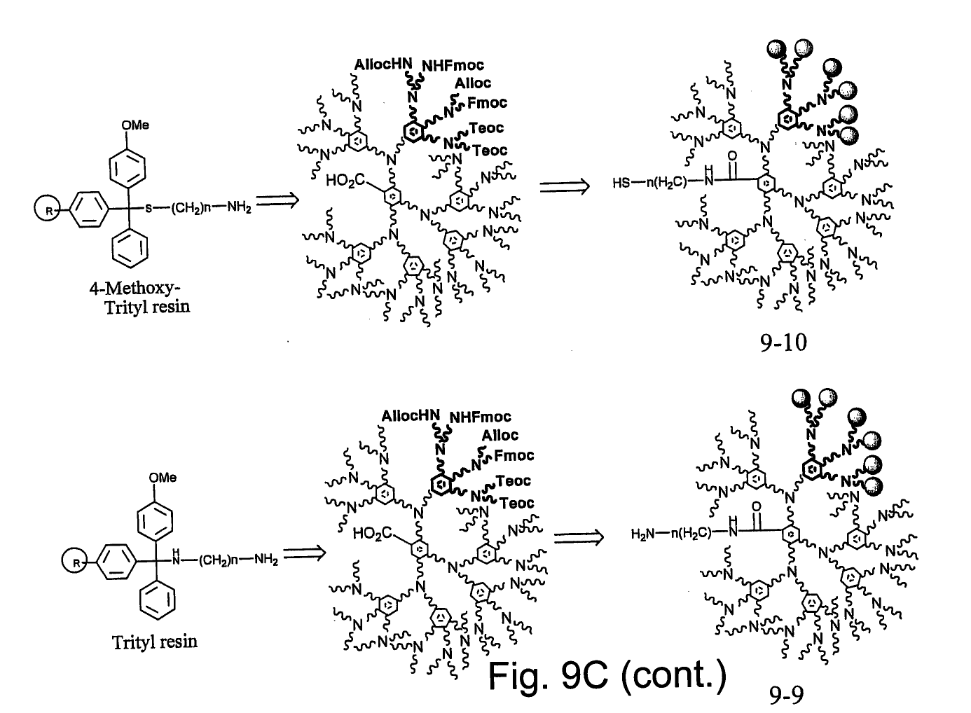

Next, Fmoc was deprotected and another preactivated drug, etoposide (p-Nitrophenyl-CO2Cl, TEA, DMAP, DCM) was coupled to afford 5-9. Cleavage from resin under mild acidic conditions led to the loaded platform 9-8 (FIG. 9A), ready to be conjugated to the carrier. The drugs are attached by primary carbamate through primary amine and by secondary carbamate through the secondary amine, differentiating drug release.

### Example 6

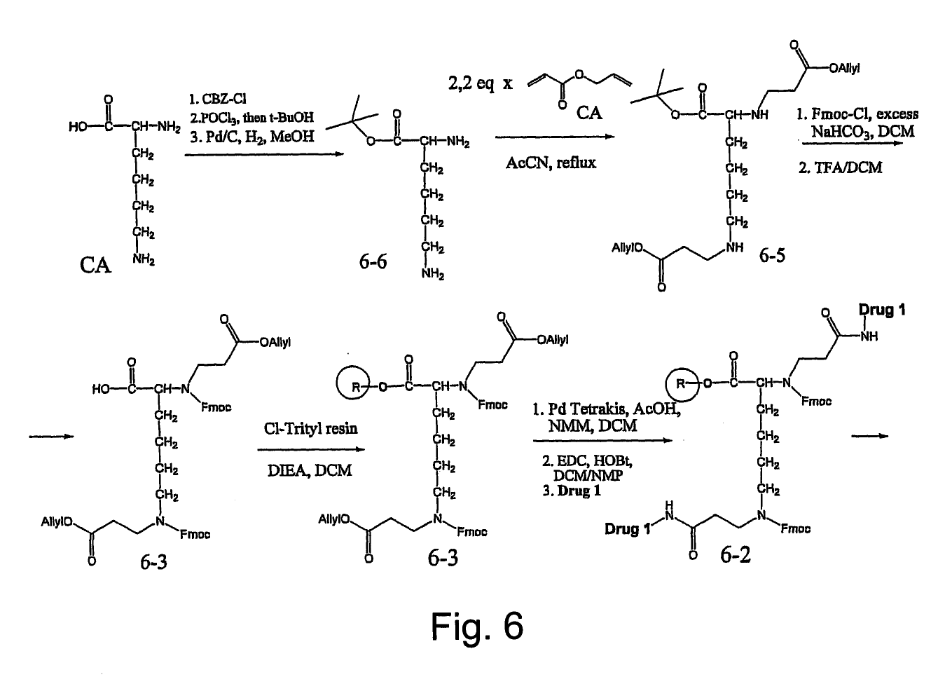

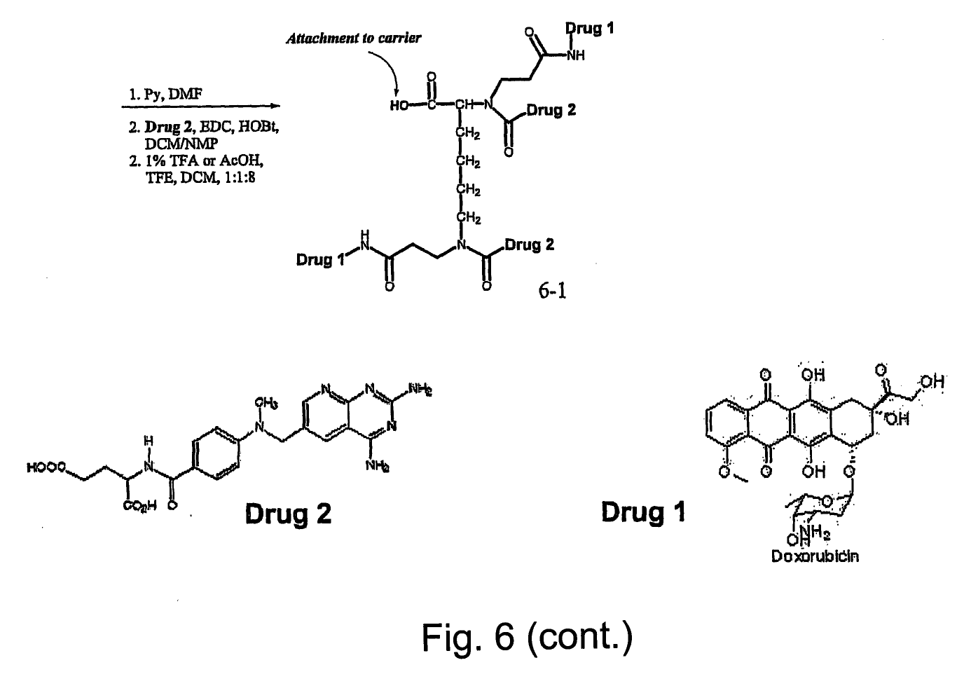

A tetravalent platform was prepared, for loading two different drugs, two molecules each, by SPOC, and the comparison of their release rates in vitro was performed. The tetra-functional platform was used for loading of acid sensitive and acid stable drugs by SPOC, and the release rates of two conjugated drugs were compared in vitro. Lys-Dialkylated platform 6-2 (see FIG. 6) was prepared and loaded with four molecules (2 doxorubicine and 2 methotextrate) by SPOC on super sensitive Cl-Trityl resin. Initially, the Fmoc, Allyl protected platform was prepared from Lys tBu ester by regiospecific double Michel addition with 2.2 eq. of allyl acrylate [Hirokazu T.: Org. Biomol. Chem. 1 (2003) 3656-62]. followed by double Fmocilation, then cleaved under very mild acidic hydrolysis. The loading of the drugs was performed on acid super sensitive solid support (Cl-Trityl resin) ending as a carboxylic acid 6-1 (FIG. 6) ready for conjugation with carrier. The drugs were attached by primary amide bond, but different amines (alpha amine vs side chain amine) differentiating the drug release. Using various side chains, absolute configurations, or linker lengths (D,L Lys, D,L-Orn, D,L-Diaminopropanoic acid and etc.) will additional variability of release rates of drugs from this platform.

### Example 7

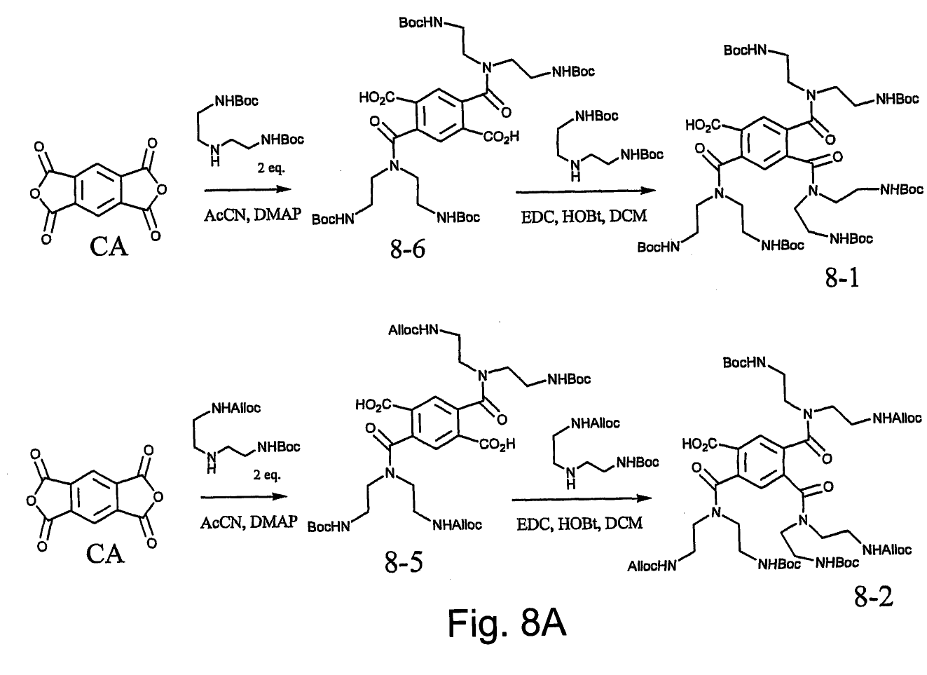

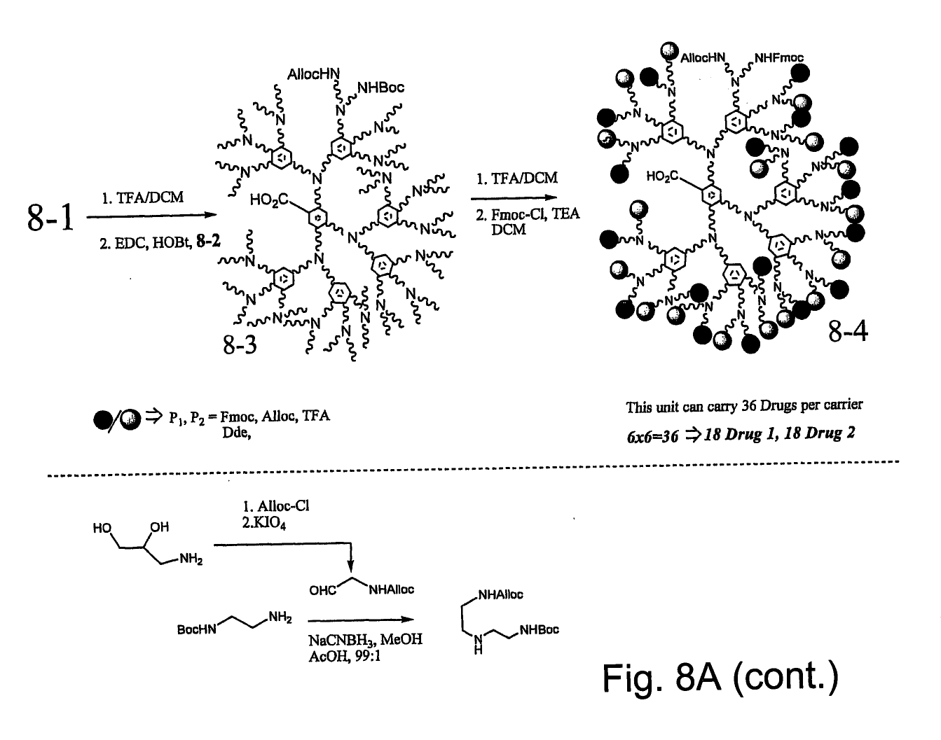

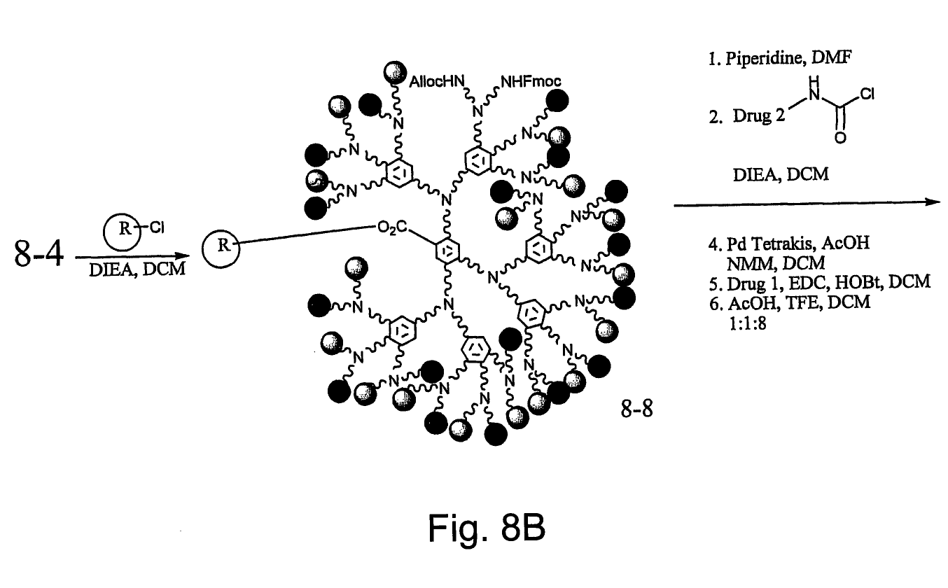

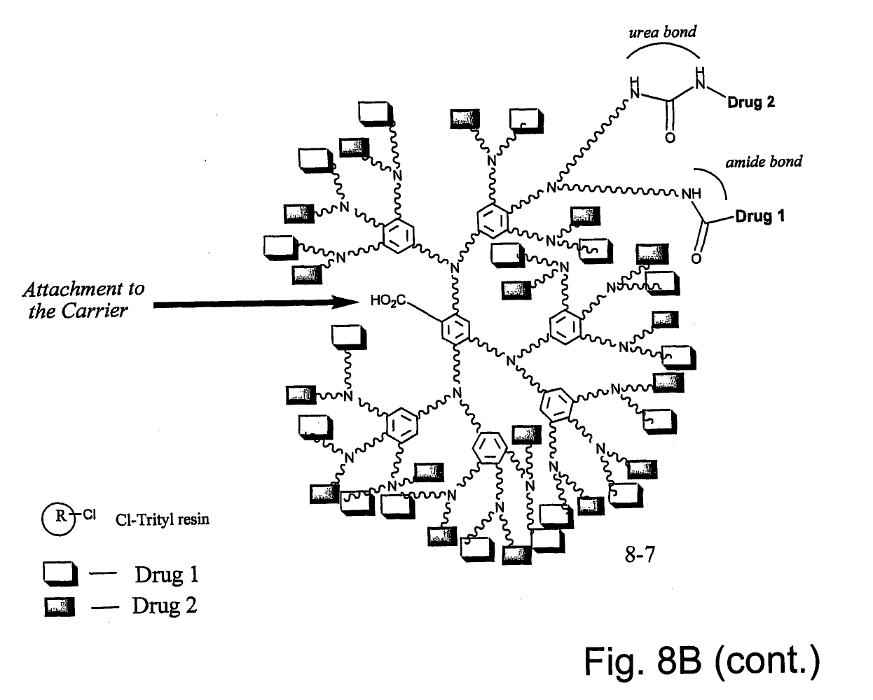

A 36-valent platform was prepared, for loading two different drugs, eighteen molecules each, by SPOC, and the comparison of their release rates in vitro was performed. The 36-functional and di-orthogonally protected platform for loading with 36 acid sensitive drug molecules by SPOC is shown in FIG. 8A and FIG. 8B. A 36 NH-Alloc/Fmoc protected platform 8-4 (FIG. 8A) was loaded with 18 molecules of doxorubicine and 18 molecules of Boc or Cbz-Melphalan by SPOC on super sensitive Cl-Trityl resin.

The Fmoc, Allyl protected platform was prepared from polymellitic anhydride. In the first step the anhydride was reacted with excess of Di-Boc triamine to produce 8-6. Additional equivalent of the di-Boc triamine lead to hexa-bocinated 8-1. Unit 8-2 was prepared by the same manner using Alloc, Boc triamine. Then, 8-1 was deprotected and submitted to the coupling with 8-2 (EDC, HOBt, DCM/AcCN) to afford 8-3, which after subsequent deprotection (TFA/DCM) and Fmoc protection led to the 36 Alloc/Fmoc Platform 8-4.

The protected platform 8-4 was loaded on Cl-Trityl resin (DCM, TEA) and pre-activated drugs are sequentially loaded through the amide and urea moieties respectively. After cleavage, the desired loaded platform 8-7 (FIG. 8B) was obtained ready for conjugation with the carrier.

### Example 8

A 36-valent platform was prepared, for loading three different drugs, twelve molecules each, by SPOC, and the comparison of their release rates in vitro was performed. The preparation of 36 functional platform with triple orthogonal protection for loading 3 different acid sensitive and other drugs by SPOC is shown in FIGS. 9A and 9B. The 36 functional platform was orthogonally protected with three protecting groups: Fmoc, Alloc and Teoc. The platform enabled to load three different drugs, 12 molecules each, yielding totally 36 molecules loaded to the platform which can be conjugated to a carrier through the free carboxylic group. The synthesis started from commercial pyromellitic anhydride with is submitted to double condensation of Boc and Alloc protected N-(2-aminoethyl)ethane-1,2-diamine. This double condensation is regioselective and isomers can be separated by flush chromatography. The obtained intermediate 9-5 (FIG. 9A) further underwent coupling with di-Teoc N-(2-aminoethyl)ethane-1,2-diamine to produce 9-4. Then, after substituting Boc by Fmoc, leaving other protecting groups untouched, the unit 9-1 is formed which already bears three pairs of functional amines orthogonally protected by Fmoc, Alloc and Teoc. In the next step the unit 9-14 was prepared in the same manner as unit 8-1 (FIG. 8A) and then loaded on the acid sensitive Cl-Trityl resin through the free carboxy group (TEA, DCM). All Teoc groups are removed by KF in DMF/H2or TBAF in THF. The unit is then coupled by standard procedure (EDC, HOBt, DCM/NMP) to afford 36 amino orthogonally tri-protected platform on solid support ready for loading of 3 different drugs.

Removal of Fmoc (Piperidine, DMF) and sequential coupling, forming amide bond with pre-made Boc or Cbz -Melphalan, yields 9-8. Removal on Alloc (Pd Tetrakis, AcOH, NMM, DCM), then forming p-nitrophenyl formate on the resin (p-NO₂-Ph-CO₂Cl, DCM) and sequential coupling, forms urea bond with doxorubicine through amine of DOX yielding 9-7. Removal of Teoc (TBAF, THF) with coupling (TEA, DCM) forms carbamate bond with pre-made Etoposide p-nitrophenyl carbonate (Etoposide, p-NO₂-Ph-COCl, DCM). After cleavage (AcOH, TFE, DCM, 1:1:8, 30 min) the 36 drug loaded platform has a free CO2H group and can be conjugated to the carrier through amine (forming amide moiety), hydroxyl (forming ester moiety) or thiol (forming thio-ester moiety).

The loading-on-the carrier moiety of platform (CO2H, in this example) can be changed to other moieties like NH2-(CH2)n-, SH—(CH2)n- or OH—(CH2)n-. The diversification of the loading end of the loaded platform can be achieved by employing different commercially available preloaded resins.

### Example 9

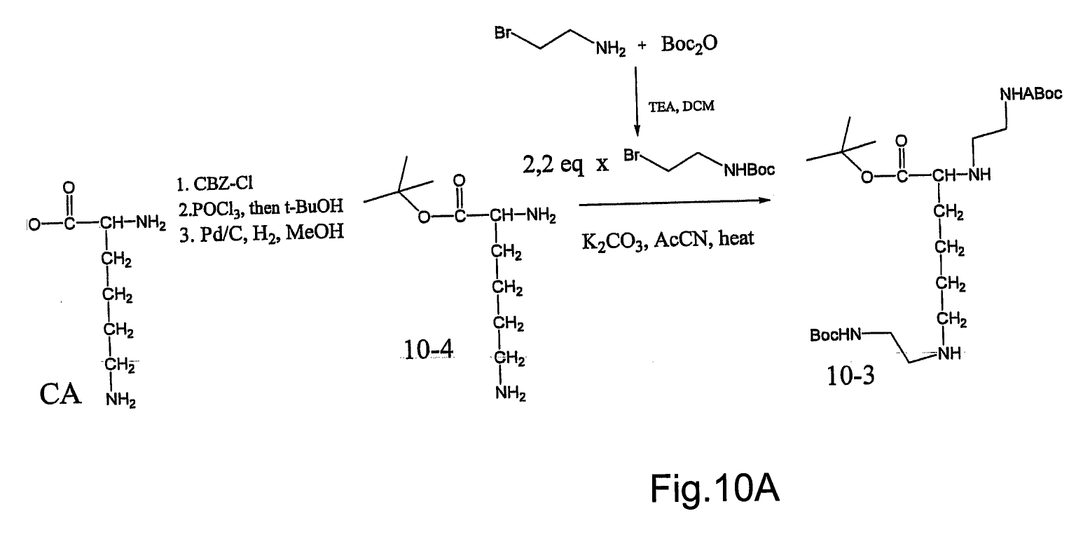

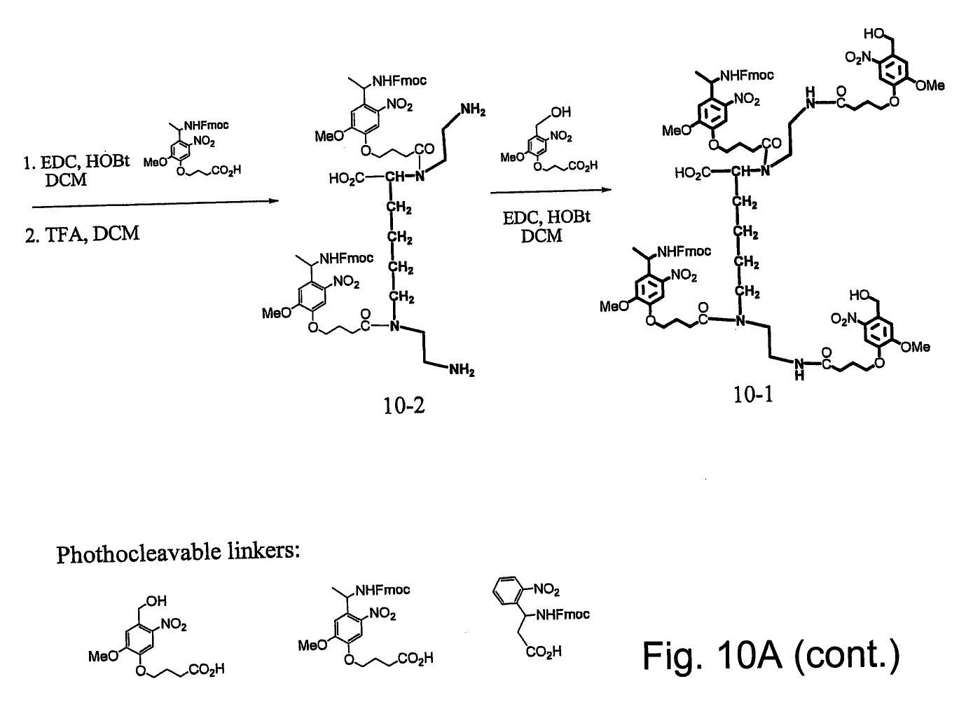

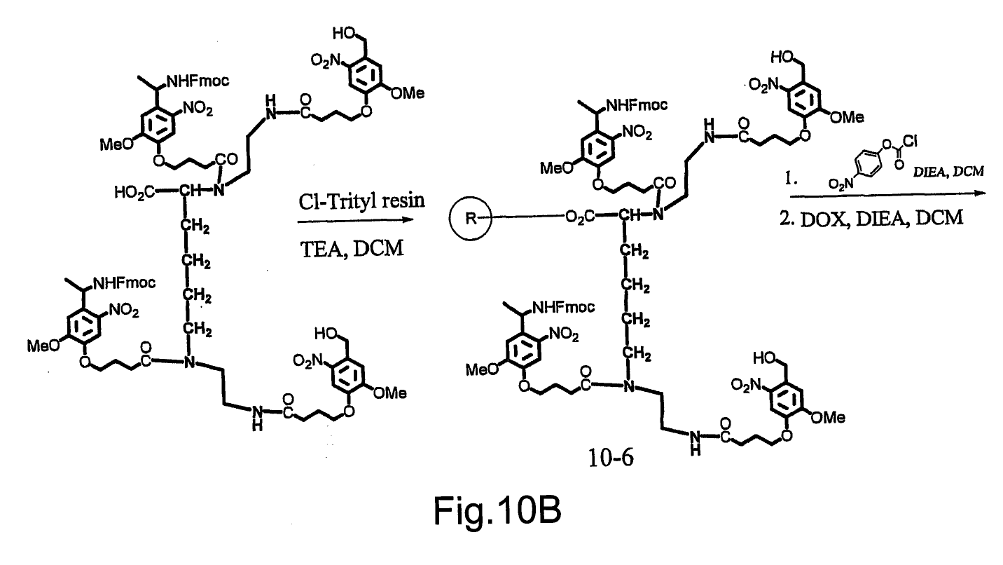

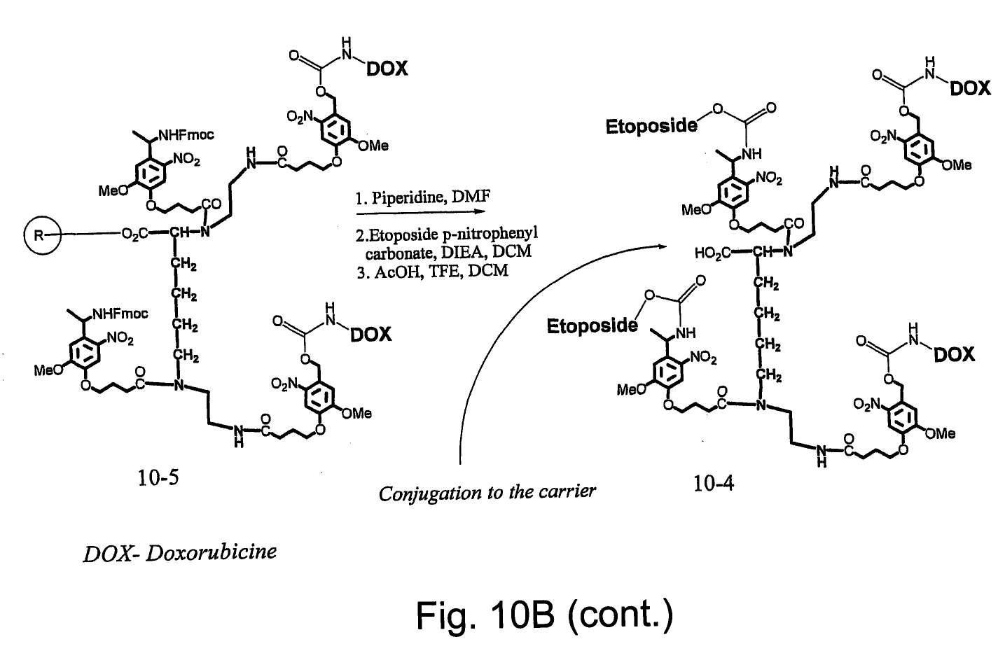

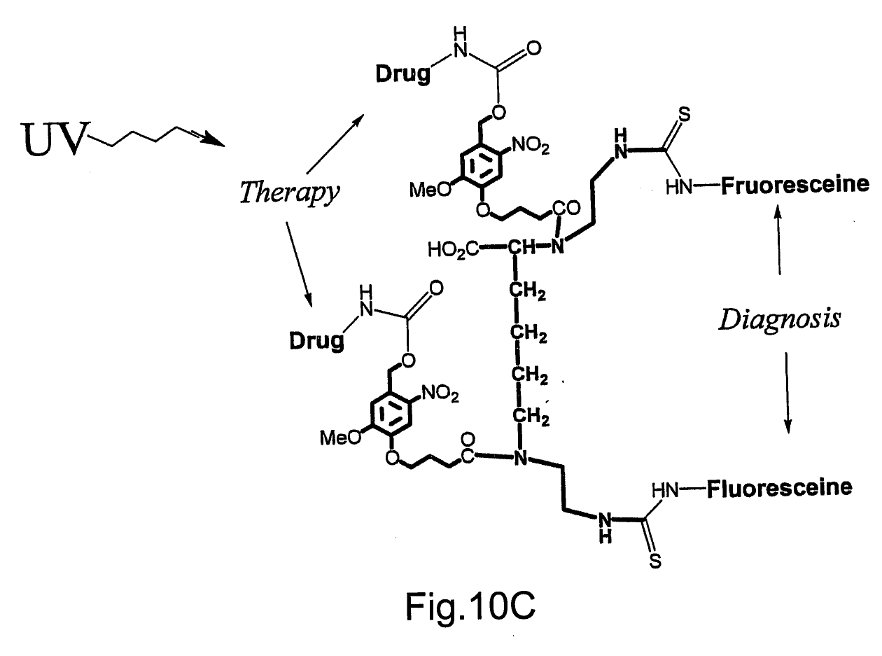

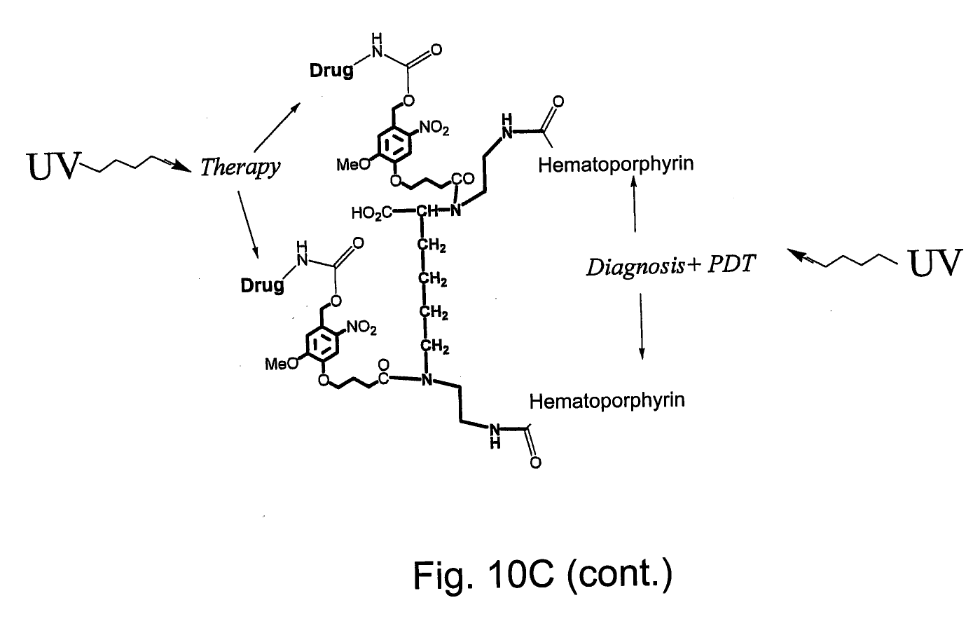

A photocleavable tetravalent platform according to the invention was prepared, for loading two different drugs, two molecules each, by SPOC, and the comparison of their release rates in vitro was performed. The platform's structure corresponds to a general structure of Formula 2 of FIG. 7. Four photocleavable linkers of two different types can bind two different drugs and differentially release under suitable conditions. The first type has Fmoc protected amino and can create ureido, amido and carbamate linkage with the conjugated drugs. The second type has free hydroxyl and is suitable for creating carbonate, carbamate, and ester linkage with conjugated drugs. The platform releases the conjugated drugs upon UV irradiation, comprising 354 nm light, another mechanism of drug release from platforms of the invention, in addition to enzymatic hydrolysis, details are shown in FIGS. 10A, 10B, and 10C.

Drugs are loaded onto the platform linked to a solid support, utilizing drugs pre-activated by p-nitrophenyl carbamate or carbonate, followed by the cleavage of the linkage between the platform and the support, exposing a free group on the platform (in this case carboxyl), available for conjugating to a carrier.

Another approach combines utilization of fluorescent label like fluoresceine (FIG. 10C) with a drug connected to the platform by a photocleavable linker. Once the platform enters the target cell, assisted by a specific carrier, the loaded platform when irradiated with UV will release the drug(s) without utilizing chemical or biological cleavage in the cell.

The drugs can de attached to the platform by photocleavable linker in combination with hematoporphyrine, using the approach of photodynamic therapy (PDT), leading to a photorelease of a drug (for example intercalating agent like melphalan) at the target.

### Example 10

A platform according to the invention, based on trihydroxybenzoic acid (THB), for loading one or two drugs was prepared. Platform 11-3 (FIG. 11A) can be loaded with nine molecules of one drug (11a in FIG. 11A) or six molecules of two different drugs (FIG. 11-b). The special case is the orthogonally protected THB platform in FIG. 11C. Platform 11-1C with nine Alloc protections is attached to the Cl-Trityl resin. The synthesis of fully Allocated nona-amino-platform 11-10 started with full alkylation of methyl 2,4,5-trihydroxybenzoate with AllocNH—CH2-CH2-Br, followed by hydrolysis.

Coupling of 11-1a or 11-1b to the methyl ester of 11-2b (EDC, HOBt, DCM) followed by hydrolysis (K2CO3, MeOH/H2O) afforded the desired 11-3, which is able to carry nine Drugs of the same type (FIG. 11A). As a part of the invention, the synthesis of fully and orthogonally tri-protected platform is described (FIG. 11A). The synthesis starts with t-butyl 3,5-dihydroxy-4-iodo benzoate leading after two sequential alkylations (KOtBu, AcCN) to the intermediate 11-7. Sonogashira coupling of the protected by Teoc (Me3Si—CH2-CH2-O—CO—) propargil amine to the 11-7 results in more advanced intermediate 11-6. Deprotection of 11-6 in TFA/DCM and consequent Fmoc protection finally desired 11-5. The platform 11-5 is fully orthogonal and can load independently three different Drugs. In FIG. 11B is described the synthesis of THB based platform for carrying six drugs: 2 different drugs, 3 molecules each).

Coupling of Teoc-L Orn(Alloc)-OH) to methyl ester 11-2b and subsequent submission to the hydrolysis affords 11-8, a new platform with double orthogonal protection (FIG. 11B). Such a platform is able to carry 6 molecules (3 of Drug1 and 3 of Drug2). As it may already noticed, the trihydroxybenzoic acid platform is versatile, varying in arm lengths, position of attachment moieties and orthogonal protection (see general structure 11 in FIG. 11B). The platform 11-9 described in FIG. 11C is more versatile and is protected by 3 orthogonally protecting groups. This platform was prepared and loaded on solid support, but also can be prepared in solution.

In case of the synthesis on solid support, the route starts with loading on the Cl-Trityl resin of fully allocated THB 11-1c from FIG. 11A. After full Alloc deprotection the versatile linker 9-4 (FIG. 9A) is coupled (EDC, HOBt, DCM) giving the fully orthogonally intermediate on the resin 11-9 and ready for loading the Drugs. Due to the full orthogonally, the drugs can be loaded by few orders. The loading of the drugs is performed while the platform is bound to solid support, utilizing premade p-nitriphenyl carbamate, carbonate derivatives or activated esters of the drugs. After loading of the drugs on activated platform, 11-9 bound to support R is cleaved under very mild acidic conditions to yield drug-loaded platform 11-11 ready for the conjugation to a carrier with a free attachment group (in this case carboxyl).

In general, preparing multifunctional platforms, including the drug loading, is preferably clone by SPOC than in solution, being rapid, convenient and effective, and further also convenient from the viewpoint of subsequent conjugation with all kinds of carriers.

### Example 11

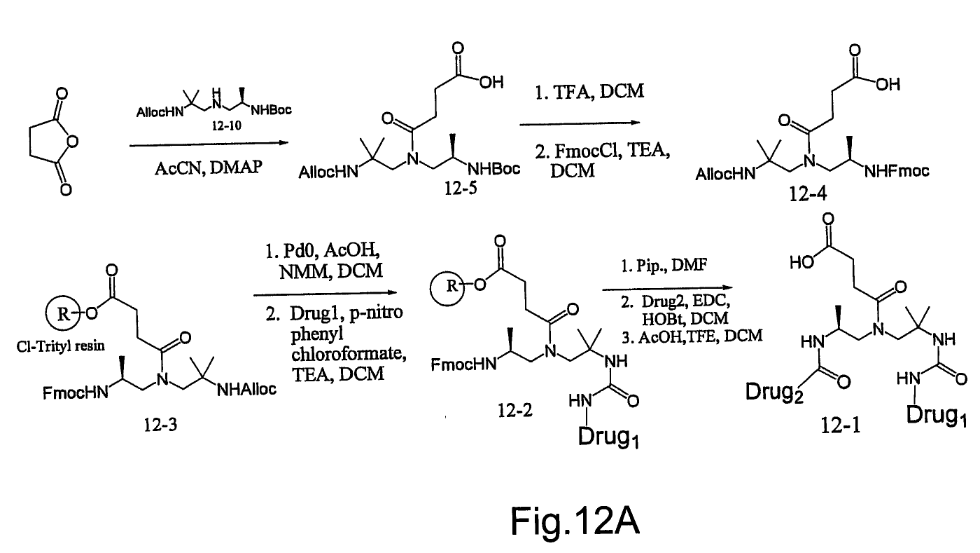

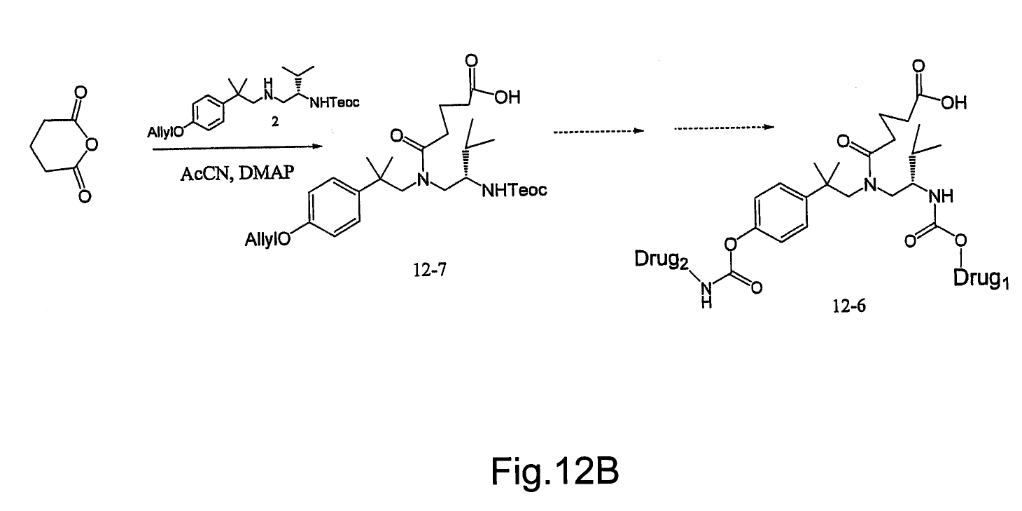

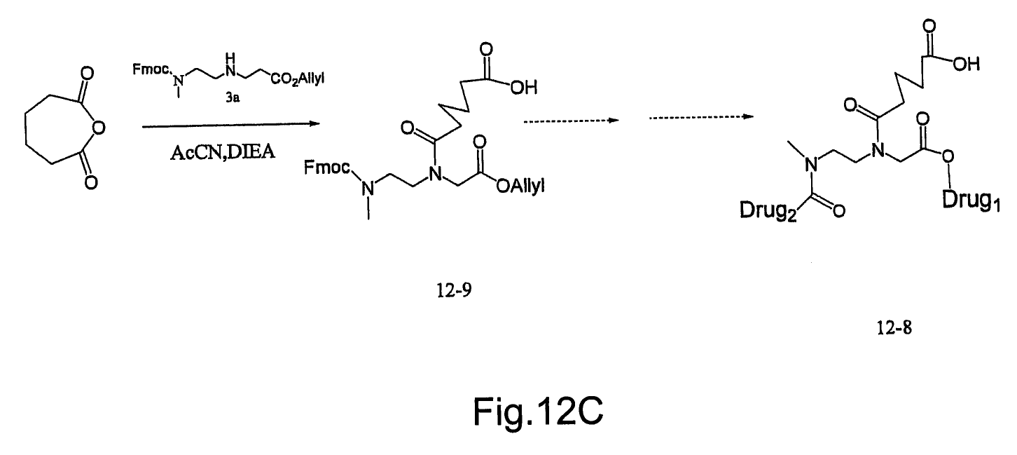

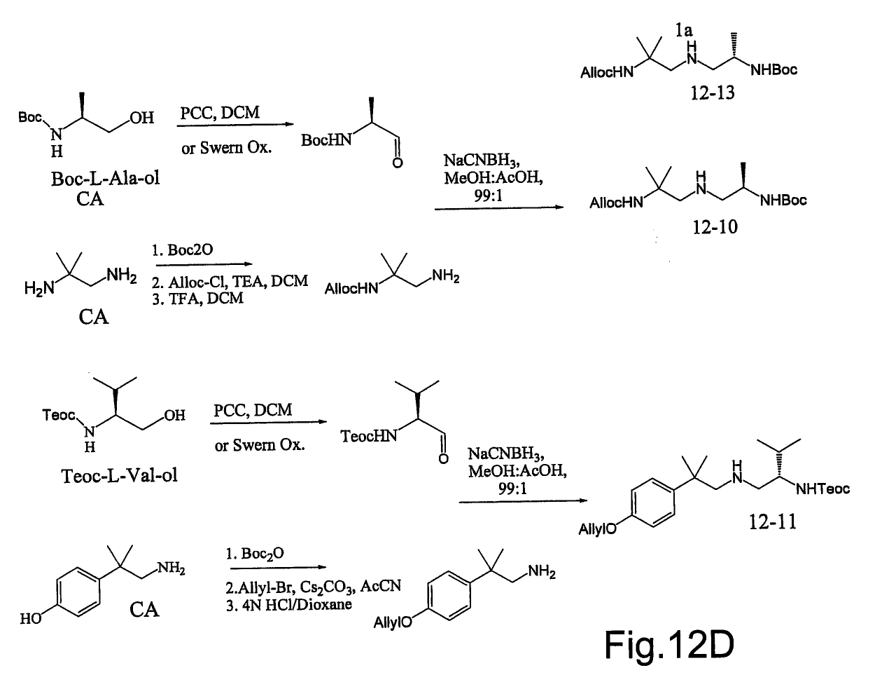

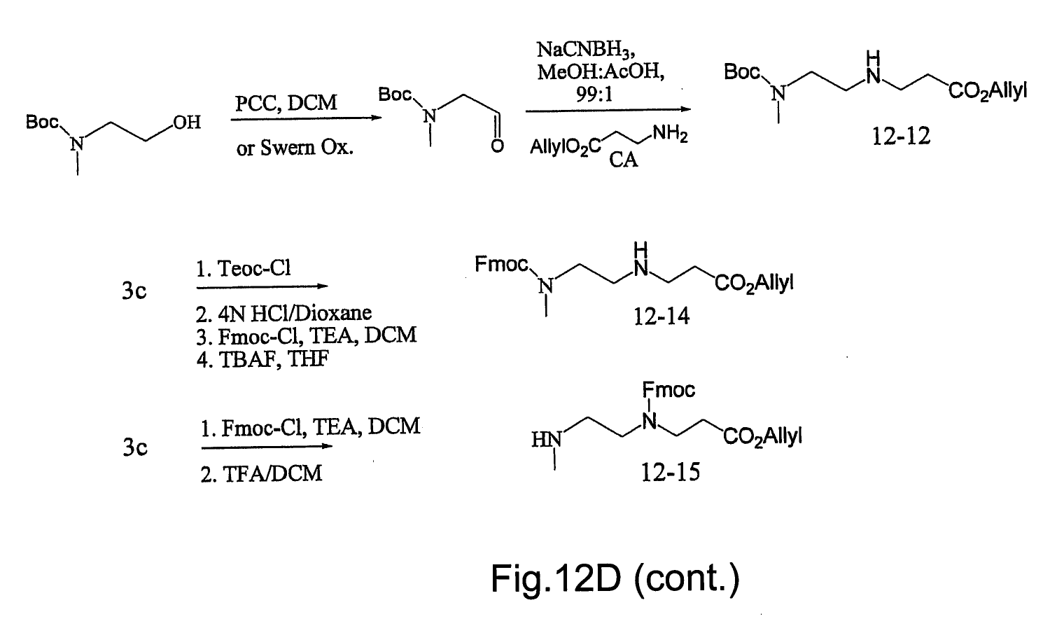

Effects of the linkers and attachment moieties on the drug release was studied. The invention relates to fine tunable release of drugs from the dendrimeric plarform. Known means of organic synthesis may be selected in creating at least two different coupling moieties in attaching at least two different drugs to the platform fro sequential, tunable, release. Some synthetic modes are shown in FIG. 12 for the preparation of a few linkers that upon reaction with cyclic anhydride provide a bifunctional orthogonally protected platform or platform intermediate.

A useful step in synthesis of the linkers is reductive alkylation of commercially available or premade aldehyde with appropriate amine to yield secondary amine derivative that will react with the cyclic anhydride [see, e.g., Gellerman G. et al.: J. Pep. Res. 57 (2001) 277]. Then, by protection/deprotection operations, the desired linkers 12-10, 12-13, 12-11, and 12-12, 12-14, and 12-15 are prepared. Next, the linkers are reacted with anhydride (FIGS. 12A, B, C) to afford bifunctional platform ready for loading on the resin. After loading, for example on Cl-Trityl resin, the drugs are coupled using standard procedure depending on linker and drug. For instance, FIG. 12A shows loading two drugs, attached to the platform by amide and urea moieties, providing 12-1. The alpha methyl groups on the linkers will cause variation in release rate in comparison with linkers that have no groups at alpha position. FIG. 12B shows two different drugs linked through two different carbamate moieties, 12-6, one moiety linked to phenol and another through amine. Preactivation of the drugs in both cases is carried out by preparing p-nitrophenyl carbamate or carbonate derivatives (p-nitrophenyl chloroformate, TEA, DCM). Alternatively, the drugs can be reacted with preactivated resin in the same way. The different linkers in FIG. 12C, comprising amide-like moiety and ester-like moiety, will affect the time release of the bound drugs in 12-8. Amide is prepared by usual coupling (EDC, HOBt, DCM/NMP or PyBrop, DIEA, NMP) and ester is prepared by Mukayama esterification.

While this invention has been described in terms of some specific examples, many modifications and variations are possible. It is therefore understood that within the scope of the appended claims, the invention may be realized otherwise than as specifically described.

1. A multifunctional platform for covalent binding of at least two different therapeutic or diagnostic agents and for their sequential release at a target site in a biological environment, said platform being a molecular structure that has
i) at least two reactive terminal groups (called attachment moieties), comprising at least two different group kinds, through which said at least two different agents are bound, forming at least two types of linkage moieties, resulting in at least two different types of cleaving kinetics under the conditions of said biological environment; and
ii) an additional terminal group (called a carrier moiety) differing from said attachment moieties, through which a recognition structure, called a carrier, is bound, wherein said carrier assists in delivering at least one of said therapeutic or diagnostic agents to said target site.

2. A multifunctional platform according to claim 1, having more than two attachment moieties of each kind.

3. A multifunctional platform according to claim 1, having at least three kinds of attachment moieties.

4. A multifunctional platform according to claim 1, having a structure selected from formulae 13-1, 13-2, 13-3, 13-4, 13-5, 13-6, 13-7, and 13-8.

5. A multifunctional platform according to claim 3, having structure 14 as follows: 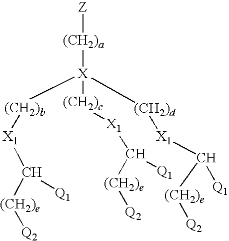
wherein X represents an entity selected from carbon atom, heterocyclic or aromatic ring selected from benzene, naphthalene, diphenyl, phenylbenzyl;
Z is a reactive group selected from —COOH, —NH₂, —NHalkyl, —OH,
—SSH, SH, and —NHNH₂; a, b, c, d, and e are integers independently selected from 1 to 5;
X₁ is selected from —NH—, —NHCO—, —CONH—, —O—, and —S—; and
Q₁ and Q₂ are groups independently selected from NHR, NHNR, COOR, OR, SR, S—SR, POₙR wherein n is 1-3, wherein R is selected from H, alkyl, aryl, and blocking groups, wherein said blocking group may be for example selected from Alloc, Fmoc, Boc, Teoc, TFA, and Dde, for NHR or NHNHR; from Acm, Trityl and s-tBu for SR or SSR, and from Me, Allyl, t-Bu, Benzyl, Dimethoxybenzyl, Fluorenemethylene for COOR, which blocking groups can be replaced by two different drug molecules, and wherein said reactive group Z couples said multifunctional platform to said carrier.

6. A multifunctional platform according to claim 1, wherein said linkage moieties comprise at least one item selected from ester, amide, secondary amide, carbamate, thiocarbamate, urea, thiourea, ether, thioether, and —S—S— group.

7. (canceled)

8. A multifunctional platform according to claim 1, wherein said sequential release of said agents comprises one or more hydrolytic enzymes present in said biological environment.

9-14. (canceled)

15. A platform according to claim 1, in which the attachment points are employed for coupling a linker comprising at least two additional attachment points, thereby enlarging the platform to a highly branched dendrimer.

16. A method for preparing the multifunctional platform of claim 1, comprising the steps of
i) providing a molecular structure comprising reactive groups of at least three different kinds, the location of the groups defining attachment points on said structure, the group kinds independently selected from —YₘPₘ, wherein Yₘ, is a radical comprising one of —NH, —O, —S, —SS, —COO, —NHNH, —N-alkyl-NH, -Ph-NH, -Ph-CH₂—NH, -Ph-O, -Ph-S, —N-alkylene, —N-cycloalkylene, or POₙ wherein n is from 1 to 3, and wherein Pₘ is a blocking group used in SPOC;
ii) contacting said structure of step i) in a solution with a resin capable of reacting with one kind of said reactive groups, thereby linking the structure through one of the attachment points to the resin and immobilizing it;
iii) contacting said immobilized structure of step ii) with at least two different drugs, or reactive derivatives of said drugs, under conditions enabling the replacement of two remaining kinds of said blocking groups, called attachment moieties, by the molecules of said drugs, thereby obtaining the immobilized platform loaded with at least two drugs; and
iv) releasing said loaded platform from the resin and binding it through said attachment point of step iii) to a carrier.

17. A method according to claim 16, wherein said Yₘ is a radical selected from the group consisting of —NH, —(CH₂)NH, —O, —(CH₂)ₙO, —S, —(CH₂)ₙS, —SS, —(CH₂)ₙSS, —COO, —(CH₂)ₙCOO, —NHNH, (CH₂)ₙNHNH, —N-alkyl-NH, —(CH₂)ₙN-alkyl-NH, -Ph-NH, (CH₂)ₙPh-NH, -Ph-CH₂—NH, -Ph-O, -Ph-S, —(CH₂)ₙPh-CH₂—NH, —N— alkylene, —(CH₂)ₙN-alkylene, —N-cycloalkylene, and —(CH₂)ₙN-cycloalkylene.

18. A method according to claim 16, wherein said Pₘ is a blocking group selected from
the group consisting of Fmoc, Alloc, Teoc, Boc, Dde, Phthalimide, Treoc, and TFA when Yₘ is a radical comprising —NH;
the group consisting of Allyl, Benzyl, Dimethoxybenzyl, Acetyl, Fluorenemethylene, t-Bu, and Trityl, when Yₘ, is a radical comprising —O;
the group consisting of S-tBu, t-Bu, Trityl, and Acm, when Yₘ, is —S; and
the group consisting of Me, Allyl, Benzyl, Dimethoxybenzyl, Fluorenemethylene, and t-Bu, when Yₘ is a radical comprising —COO.

19. A method according to claim 16, wherein said carrier is a molecular structure covalently linked to said platform, assisting in delivering a therapeutic or diagnostic agent to the desired site of action in a tissue, either targeting said tissue or stabilizing said agents during their transport to the tissue.

20. A method according to claim 16, wherein said molecular structure is a molecule or a part thereof selected from protein, peptide, phospholipid, polysaccharide, nucleic acid or a structural mimic thereof, such as a peptide nucleic acid (PNA) and biodegradable polymer.

21. A method according to claim 16, wherein said molecular structure is a molecule or a part thereof having high affinity to a tissue to be treated.

22. A method according to claim 16, wherein said molecular structure recognizes or is recognized by a treated tissue.

23. A method according to claim 16, wherein said molecular structure is a molecule or a part thereof that interacts with a regulation cascade in vivo, thereby initiating processes supporting intended therapeutic goals.

24. A method according to claim 16, farther comprising a step of coupling to the existing attachment points a linker comprising at least two additional attachment points, thereby enlarging the platform to a highly branched dendrimer with higher loading capacity.
